(19) United States
(12) Patent Application Publication
(10) Pub. No.: US 2018/0340180 A1
(43) Pub. Date: Nov. 29, 2018

(54) DOMINANT-NEGATIVE GENETIC MANIPULATION TO MAKE LOW-NICOTINE TOBACCO PRODUCTS

(71) Applicant: 22nd Century Limited, LLC, Williamsville, NY (US)

(72) Inventor: Paul Rushton, Williamsville, NY (US)

(73) Assignee: 22nd Century Limited, LLC, Williamsville, NY (US)

(21) Appl. No.: 15/983,704

(22) Filed: May 18, 2018

Related U.S. Application Data
(60) Provisional application No. 62/508,877, filed on May 19, 2017.

(51) Int. Cl. C12N 15/82

(52) U.S. Cl. CPC C12N 15/8243; C12N 15/8237; C12N 15/8222

(57) ABSTRACT

The present technology provides dominant negative forms of transcription factors for modifying nicotine biosynthesis and nucleic acid molecules that encode such dominant negative transcription factors. Also provided are methods of using these nucleic acids to modulate nicotine production in plants and for producing plants and plant cells having reduced nicotine content.

### CROSS-REFERENCE TO RELATED APPLICATION

This application claims the benefit of and priority to U.S. Provisional Patent Application No. 62/508,877, filed May 19, 2017, the content of which is incorporated herein by reference in its entirety.

### TECHNICAL FIELD

The present technology relates generally to the use of dominant negative forms of transcription factors for modifying nicotine biosynthesis, nucleic acid molecules that encode such dominant negative transcription factors, and methods of using these nucleic acids to modulate nicotine production in plants and for producing plants and plant cells having reduced nicotine content.

### BACKGROUND

The following description is provided to assist the understanding of the reader. None of the information provided or references cited is admitted to be prior art.

The production of tobacco with decreased levels of nicotine is of interest given the addictiveness of nicotine and nicotine products, namely cigarettes. Additionally, tobacco plants with extremely low or no nicotine production, are attractive as recipients for transgenes expressing commercially valuable products such as pharmaceuticals, cosmetic components, or food additives. Various processes have been designed for the removal of nicotine from tobacco. However, most of these processes remove other ingredients from tobacco in addition to nicotine, thereby adversely affecting the tobacco. Classical crop breeding techniques have produced tobacco plants with lower levels of nicotine (approximately 8%) than that found in wild-type tobacco plants. Tobacco plants and tobacco having even further reductions in nicotine content are desirable.

Nicotine, a pyrrolidine alkaloid, is among the most abundant alkaloids produced in Nicotiana spp., and is synthesized in the roots and then translocates through the plant vascular system to the leaves and other aerial tissues where it serves as a defensive compound against herbivores. The enzymes involved in the major steps of nicotine biosynthetic pathway in Nicotiana tabacum (tobacco) have been characterized. Major nicotine biosynthetic pathway genes include aspartate oxidase (AO), quinolinate synthase (QS), quinolinic acid phosphoribosyltransferease (QP7), ornithine decarboxylase (ODC), arginine decarboxylase (ADC), putrescine N-methyltransferase (PMT), N-methylputrescine oxidase (MPO), diamine oxidase (DAO), an isoflavone reductase like protein, A622, and NBB1. The biosynthesis of nicotine involves pyrrolidine ring formation, pyridine ring formation, and the coupling of both rings. The enzymes ADC, ODC, PMT, MPO, and DAO are involved in the formation of the pyrrolidine ring (FIG. 1). AO, QS, and QPT are responsible for the biosynthesis of the pyridine ring. These three enzymes are also involved in the synthesis of nicotinic acid dinucleotide (NAD). A622 and berberine bridge enzyme-like protein (BBL) are required for nicotine ring coupling. Data from a range of studies revealed that the regulation of nicotine biosynthesis involves a range of proteins, hormones, kinases, and transcription factors.

One approach for reducing the level of a biological product, such as nicotine, is to reduce the amount of a required enzyme in the biosynthetic pathway leading to the product. This may be accomplished by altering the expression of the gene encoding the enzyme itself or of the transcriptional factor that controls its transcription. Transcription factors are DNA-binding proteins that interact with other transcriptional regulators to recruit or block access of RNA polymerases to the DNA template. The regulation of gene expression at the level of transcription is a major point of control in many biological processes, including plant metabolism and nicotine biosynthesis. Although there are several known techniques for altering gene expression, including antisense technology, site-directed mutagenesis, and co-suppression, these technologies require an accurate knowledge of the structure of the gene or transcription factor to allow the antisense sequence to specifically hybridize with the gene sequence or to allow the mutation to only affect some properties of the protein. Other limitations of these methods include redundancy in genes and/or functions whereby other related genes that are not affected by these targeted technologies conceal the expression of the target gene. As a result, the observed phenotype is wild-type rather than the expected mutant. Accordingly, there is a need to develop methods that circumvent the problems associated with the aforementioned techniques and that can be used for the modification of tobacco nicotine levels in plants, including transgenic tobacco plants, cell lines, and derivatives thereof.

### SUMMARY

Disclosed herein are methods and compositions for reducing nicotine biosynthesis in plants.

In one aspect, the present disclosure provides a chimeric nucleic acid construct comprising at least one transcription repressor sequence in translational fusion with at least one transcription factor sequence encoding for a transcription factor or fragment thereof that positively regulates nicotine biosynthesis, wherein the chimeric nucleic acid construct is operably linked to a heterologous nucleic acid. In some embodiments, the transcription repressor sequence encodes for a transcription repressor selected from the group consisting of: Engrailed (En²⁹⁸) (SEQ ID NO: 11), SRDX (SEQ ID NO: 12), AtERF4 (SEQ ID NO: 13), NtERF3 (SEQ ID NO: 15), SUPERMAN (SEQ ID NO: 17), LEUNIG (SEQ ID NO: 18), and SEUSS (SEQ ID NO: 19). In some embodiments, the transcription factor sequence encoding for a transcription factor or fragment thereof that positively regulates nicotine biosynthesis is selected from the group consisting of: (a) the nucleic acid sequence of SEQ ID NO: 1 (NtERF1), SEQ ID NO: 2 (NtERF5), SEQ ID NO: 3 (NtERF10), SEQ ID NO: 4 (NtERF32), SEQ ID NO: 5 (NtERF221), SEQ ID NO: 6 (NtERF241), SEQ ID NO: 7 (NtMYC1a), SEQ ID NO: 8 (NtMYC1b), SEQ ID NO: 9 (NtMYC2a), or SEQ ID NO: 10 (NtMYC2b); and (b) a nucleic acid sequence that is at least about 80% identical to the nucleic acid sequence of (a), and which encodes for a protein comprising at least one DNA binding domain that positively regulates nicotine biosynthesis.

In one aspect, the present disclosure provides an expression vector comprising a chimeric nucleic acid construct comprising at least one transcription repressor sequence in translational fusion with at least one transcription factor sequence encoding for a transcription factor or fragment thereof that positively regulates nicotine biosynthesis, wherein the chimeric nucleic acid construct is operably linked to a heterologous nucleic acid, wherein the operably linked heterologous nucleic acid comprises one or more control sequences suitable for directing expression in a Nicotiana host cell.

In one aspect, the present disclosure provides a Nicotiana host cell transformed with a chimeric nucleic acid construct comprising at least one transcription repressor sequence in translational fusion with at least one transcription factor sequence encoding for a transcription factor or fragment thereof that positively regulates nicotine biosynthesis, wherein the chimeric nucleic acid construct is operably linked to a heterologous nucleic acid.

In one aspect, the present disclosure provides a genetically engineered nicotinic alkaloid-producing Nicotiana plant comprising a cell comprising a chimeric nucleic acid construct comprising at least one transcription repressor sequence in translational fusion with at least one transcription factor sequence encoding for a transcription factor or fragment thereof that positively regulates nicotine biosynthesis, wherein the chimeric nucleic acid construct is operably linked to a heterologous nucleic acid. In some embodiments, the plant is a Nicotiana tabacum plant.

In one aspect, the present disclosure provides seeds from a genetically engineered nicotinic alkaloid-producing Nicotiana plant comprising a cell comprising a chimeric nucleic acid construct comprising at least one transcription repressor sequence in translational fusion with at least one transcription factor sequence encoding for a transcription factor or fragment thereof that positively regulates nicotine biosynthesis, wherein the seeds comprise the chimeric nucleic acid construct.

In one aspect, the present disclosure provides a tobacco product comprising a genetically engineered nicotinic alkaloid-producing Nicotiana plant comprising a cell comprising a chimeric nucleic acid construct comprising at least one transcription repressor sequence in translational fusion with at least one transcription factor sequence encoding for a transcription factor or fragment thereof that positively regulates nicotine biosynthesis.

In one aspect, the present disclosure provides a method for reducing nicotine in a Nicotiana plant, comprising: (a) introducing into a Nicotiana plant an expression vector comprising a chimeric nucleic acid construct comprising, in the 5′ to 3′ direction, a promoter suitable for directing expression in a Nicotiana plant cell operably linked to at least one transcription repressor sequence in translational fusion with at least one transcription factor sequence encoding for a transcription factor or fragment thereof that positively regulates nicotine biosynthesis; and (b) growing the plant under conditions which allow for the expression of the chimeric nucleic acid; wherein expression of the chimeric nucleic acid results in production of a dominant negative form of the transcription factor and the plant having a reduced nicotine content as compared to a non-transformed control plant grown under similar conditions. In some embodiments of the methods provided herein, the transcription repressor sequence encodes for transcription repressor selected from the group consisting of: Engrailed (En²⁹⁸) (SEQ ID NO: 11), SRDX (SEQ ID NO: 12), AtERF4 (SEQ ID NO: 13), NtERF3 (SEQ ID NO: 15), SUPERMAN (SEQ ID NO: 17), LEUNIG (SEQ ID NO: 18), and SEUSS (SEQ ID NO: 19). In some embodiments of the methods provided herein, the transcription factor that positively regulates nicotine biosynthesis is selected from the group consisting of: (a) the nucleic acid sequence of SEQ ID NO: 1 (NtERF1), SEQ ID NO: 2 (NtERF5), SEQ ID NO: 3 (NtERF10), SEQ ID NO: 4 (NtERF32), SEQ ID NO: 5 (NtERF221), SEQ ID NO: 6 (NtERF241), SEQ ID NO: 7 (NtMYC1a), SEQ ID NO: 8 (NtMYC1b), SEQ ID NO: 9 (NtMYC2a), or SEQ ID NO: 10 (NtMYC2b); and (b) a nucleic acid sequence that is at least 80% identical to the nucleic acid sequence of (a), and which encodes for a protein comprising at least one DNA binding domain that positively regulates nicotine biosynthesis. In some embodiments, the present disclosure provides a genetically engineered Nicotiana plant produced by the method, wherein the plant has reduced expression of nicotine as compared to a control plant. In some embodiments, the present disclosure provides a product comprising the genetically engineered plant or portions thereof, wherein the product has a reduced nicotine content as compared to a product produced from a control plant. In some embodiments, the product is a reduced-nicotine tobacco product selected from the group consisting of cigarette tobacco, reconstituted tobacco, cigar tobacco, pipe tobacco, cigarettes, cigars, chewing tobacco, snuff, snus, and lozenges. In some embodiments, the present disclosure provides seeds from the genetically engineered Nicotiana plant produced by the methods described herein, wherein the seeds comprise the chimeric nucleic acid construct.

In one aspect, the present disclosure provides a genetically engineered Nicotiana plant having reduced nicotine content as compared to a non-transformed control plant, wherein the plant comprises plant cells comprising: a chimeric nucleic acid construct comprising, in the 5′ to 3′ direction, a promoter suitable for directing expression in the Nicotiana plant cell operably linked to at least one transcription repressor sequence in translational fusion with at least one transcription factor sequence encoding for a transcription factor or fragment thereof that positively regulates nicotine biosynthesis, wherein expression of the chimeric nucleic acid results in production of a dominant negative form of the transcription factor and the plant having a reduced nicotine content as compared to a non-transformed control plant grown under similar conditions. In some embodiments, the transcription repressor sequence encodes for a transcription repressor selected from the group consisting of: Engrailed (En²⁹⁸) (SEQ ID NO: 11), SRDX (SEQ ID NO: 12), AtERF4 (SEQ ID NO: 13), NtERF3 (SEQ ID NO: 15), SUPERMAN (SEQ ID NO: 17), LEUNIG (SEQ ID NO: 18), and SEUSS (SEQ ID NO: 19). In some embodiments, the transcription factor that positively regulates nicotine biosynthesis is selected from the group consisting of: (a) the nucleic acid sequence of SEQ ID NO: 1 (NtERF1), SEQ ID NO: 2 (NtERF5), SEQ ID NO: 3 (NtERF10), SEQ ID NO: 4 (NtERF32), SEQ ID NO: 5 (NtERF221), SEQ ID NO: 6 (NtERF241), SEQ ID NO: 7 (NtMYC1a), SEQ ID NO: 8 (NtMYC1b), SEQ ID NO: 9 (NtMYC2a), or SEQ ID NO: 10 (NtMYC2b); and (b) a nucleic acid sequence that is at least about 80% identical to the nucleic acid sequence of (a), and which encodes for a protein comprising at least one DNA binding domain that positively regulates nicotine biosynthesis. In some embodiments, the present disclosure provides a product comprising the genetically engineered plant or portions thereof, wherein the product has a reduced nicotine content as compared to a product produced from a control plant. In some embodiments, the product is a reduced-nicotine tobacco product selected from the group consisting of cigarette tobacco, reconstituted tobacco, cigar tobacco, pipe tobacco, cigarettes, cigars, chewing tobacco, snuff, snus, and lozenges. In some embodiments, the present disclosure provides seeds from the genetically engineered Nicotiana plant, wherein the seeds comprise the chimeric nucleic acid construct.

In one aspect, the present disclosure provides a method of making a genetically engineered Nicotiana plant cell having reduced nicotine content, the method comprising: introducing into a Nicotiana plant cell an expression vector comprising a chimeric nucleic acid construct comprising, in the 5′ to 3′ direction, a promoter suitable for directing expression in a Nicotiana plant cell operably linked to at least one transcription repressor sequence in translational fusion with at least one transcription factor sequence encoding for a transcription factor or fragment thereof that positively regulates nicotine biosynthesis; wherein expression of the chimeric nucleic acid results in production of a dominant negative form of the transcription factor and the plant cell having a reduced nicotine content as compared to a non-transformed control plant cell. In some embodiments, the transcription repressor sequence encodes for transcription repressor selected from the group consisting of: Engrailed (En²⁹⁸) (SEQ ID NO: 11), SRDX (SEQ ID NO: 12), AtERF4 (SEQ ID NO: 13), NtERF3 (SEQ ID NO: 15), SUPERMAN (SEQ ID NO: 17), LEUNIG (SEQ ID NO: 18), and SEUSS (SEQ ID NO: 19). In some embodiments, the transcription factor that positively regulates nicotine biosynthesis is selected from the group comprising of: (a) the nucleic acid sequence of SEQ ID NO: 1 (NtERF1), SEQ ID NO: 2 (NtERF5), SEQ ID NO: 3 (NtERF10), SEQ ID NO: 4 (NtERF32), SEQ ID NO: 5 (NtERF221), SEQ ID NO: 6 (NtERF241), SEQ ID NO: 7 (NtMYC1a), SEQ ID NO: 8 (NtMYC1b), SEQ ID NO: 9 (NtMYC2a), or SEQ ID NO: 10 (NtMYC2b); and (b) a nucleic acid sequence that is at least about 80% identical to the nucleic acid sequence of (a), and which encodes for a protein comprising at least one DNA binding domain that positively regulates nicotine biosynthesis. In some embodiments, the Nicotiana plant cell is Nicotiana tabacum.

The technologies described and claimed herein have many attributes and embodiments including, but not limited to, those set forth or described or referenced in this brief summary. It is not intended to be all-inclusive and the inventions described and claimed herein are not limited to or by the features or embodiments identified in this brief summary, which is included for purposes of illustration only and not restriction. Additional embodiments may be disclosed in the detailed description below.

### BRIEF DESCRIPTION OF THE DRAWINGS

FIG. 1 is a schematic diagram depicting the nicotine biosynthesis pathway. The abbreviations are: AO=aspartate oxidase, QS=quinolinate synthase, QPT=quinolinate phosphoribosyltransferase, ODC=ornithine decarboxylase, PMT=putrescine N-methyltransfrease, DAO=diamine oxidase, and BBL=berberine bridge enzyme-like protein.

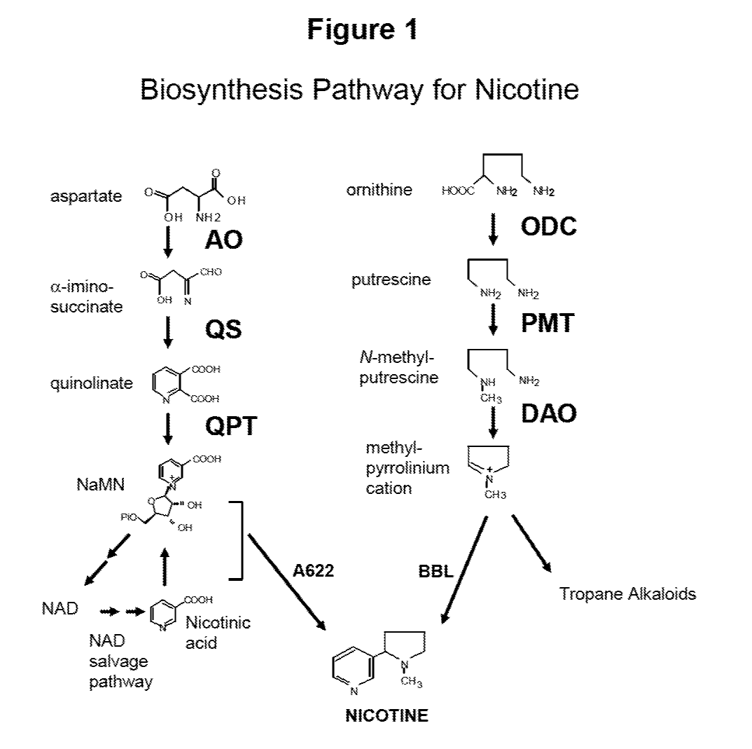

### DETAILED DESCRIPTION

### I. Introduction

Several transcription factors and putative transcription factors that positively regulate nicotine biosynthesis have been identified. Illustrative, non-limiting examples of such transcription factors are shown in Table 1.

TABLE 1
Nicotine Biosynthetic Pathway Transcription Factors

| Transcription Factor | Function | Sequence | Source(s) |
|---|---|---|---|
| NtERF1 | Biotic and abiotic | GenBank Accession No.: | Ohme-Takagi & |
| | stress. | D38123.1 | Shinshi, Plant Cell, |
| | | (SEQ ID NO: 1) | 7(2): 173-182 (1995). |
| NtERF5/NtERF121 | Positively regulates | GenBank Accession No.: | Fischer & Droge- |
| | NtPMT expression. | AY655738.1 | Laser, Mol. Plant |
| | | (SEQ ID NO: 2) | Mircrobe Interact., |
| | | | 77(10): 1162-1171 |
| | | | (2004). |
| NtERF10/JAP1 | Positively regulates | GenBank Accession No.: | WO2003097790. |
| | expression of the | CQ808845.1 | Goossens, A., et al., |
| | NtPMT2 promoter. | (SEQ ID NO: 3) | Proc. Natl Acad. Sci. |
| | | | USA, 100: 8595-8600 |
| | | | (2003). |
| | | | De Sutler, V., et al., |
| | | | Plant J. 44: 1065- |
| | | | 1076 (2005). |
| | | | Sears et al., Plant |
| | | | Mol. Biol. 84: 49-66 |
| | | | (2014). |
| NtERF32/EREBP2/ERF2 | Positively regulates | GenBank Accession No.: | Ohme-Takagi & |
| | NtPMT, NtQPT2, | D38126.1 | Shinshi, Plant Cell, |
| | NtODC1, and | (SEQ ID NO: 4) | 7(2): 173-182 (1995). |
| | NtA622 expression. | | (EREBP2) |
| | | | Sears et al., Plant |
| | | | Mol. Biol. 84: 49-66 |
| | | | (2014). |
| NtERF221/ORC1 | Positively regulates | GenBank Accession No.: | Goossens, A., et al., |
| | expression of the | CQ808982.1 | Proc. Natl Acad. Sci. |
| | NtPMT2 promoter. | (SEQ ID NO: 5) | USA, 100: 8595-8600 |
| | | | (2003). |
| | | | De Sutler, V., et al., |
| | | | Plant J. 44: 1065- |
| | | | 1076 (2005). |
| | | | Sears et al., Plant |
| | | | Mol. Biol. 84: 49-66 |
| | | | (2014). |
| NtERF241 | Predicted to | No published sequence as | Unpublished |
| | positively regulate | NtERF241. Present only as | |
| | expression of | PREDICTED: Nicotiana | |
| | nicotine biosynthetic | tabacum ethylene-responsive | |
| | pathway genes | transcription factor 2 | |
| | including NtPMT | (LOC107791664), mRNA | |
| | and NtQPT. | Sequence ID: | |
| | | XM_016613760.1 | |
| | | (SEQ ID NO: 6) | |
| NtMYC1a | Positively regulates | GenBank Accession No.: | Todd et al, Plant |
| | nicotinic alkaloid | GQ859158.1 | Journal 62: 589-600 |
| | biosynthesis. | (SEQ ID NO: 7) | (2010). |
| | | | Li et al., Frontiers in |
| | | | Plant Science, |
| | | | 7(582): 1-11 (2016). |
| | | | Zhang et al., Mol |
| | | | Plant., 5(1): 73-84 |
| | | | (2012). |
| NtMYC1b | Positively regulates | GenBank Accession No.: | Todd et al., Plant |
| | nicotinic alkaloid | GQ859159.1 | Journal 62: 589-600 |
| | biosynthesis. | (SEQ ID NO: 8) | (2010). |
| | | | Li et al., Frontiers in |
| | | | Plant Science, |
| | | | 7(582): 1-11 (2016). |
| | | | Zhang et al., Mol |
| | | | Plant., 5(1): 73-84 |
| | | | (2012). |
| NtMYC2a | Positively regulates | GenBank Accession No.: | Wang et al., Scientific |
| | nicotinic alkaloid | GQ859160 | Reports, 5: 17360 |
| | biosynthesis. | (SEQ ID NO: 9) | (2015). |
| | | | Todd et al., Plant |
| | | | Journal, 62: 589-600 |
| | | | (2010). |
| | | | Li et al., Frontiers in |
| | | | Plant Science, |
| | | | 7(582): 1-11 (2016). |
| NtMYC2b | Positively regulates | GenBank Accession No.: | Wang et al., Scientific |
| | nicotinic alkaloid | GQ859161 | Reports, 5: 17360 |
| | biosynthesis. | (SEQ ID NO: 10) | (2015). |
| | | | Todd et al., Plant |
| | | | Journal, 62: 589-600 |
| | | | (2010). |
| | | | Li et al., Frontiers in |
| | | | Plant Science, |
| | | | 7(582): 1-11 (2016). |

The majority of transcription factors are composed of a DNA-binding domain, a protein interaction surface, and a transcriptional control domain, which can activate or repress target gene activity. In animal cells, repressors, in which a DNA binding domain or a transcription factor is fused to repressor domain, have been used for the targeted repression of genes of interest. See, e.g., Bädiani et al., Genes Dev. 8:770-782 (1994); Beerli et al., Proc. Natl. Acad. Sci. 95:14628-14633 (1998); de Haan et al., J. Biol. Chem. 275:13493-13501 (2000); Joh et al., Development 121:366-377 (1995). Studies have shown that plant transcription factors can be efficiently reprogrammed to have dominant-negative functions through the use of chimeric repressor silencing technology (CRES-T), which involves fusion of at least one DNA binding domain of the transcription factor to transcriptional repressor domains, such as the ENGRAILED (En) homeodomain protein from Drosophila or an ERF-associated amphiphilic repression (EAR) motif, to effectively suppress their target genes with resultant loss-of-function phenotypes. See, e.g., Markel et al., Nucleic Acids Research 30(21):4709-4719 (2003); Hiratsu et al., The Plant Journal 34:733-739 (2003).

The present technology encompasses nucleic acid constructs and methods for producing transgenic tobacco plants and cells having decreased levels of nicotine relative to non-transformed control plants. Such methods include the expression of one or more nicotine biosynthetic pathway transcription factor-repressor domain fusion proteins, which have the capacity to reduce or eliminate the expression of one or more enzymes of the nicotine biosynthetic pathway.

The present technology also provides chimeric nucleic acid molecules comprising a nicotine biosynthetic pathway transcription factor coding sequence in transcriptional fusion with a repressor domain, and vectors comprising those recombinant nucleic acid molecules, as well as transgenic plant cells and plants transformed with those nucleic acid molecules and vectors. The present technology also provides for transgenes that are capable of reducing expression of at least one endogenous nicotine biosynthetic pathway gene in the plant cell by an amount sufficient to reduce nicotine levels in the transgenic plant cell. This transgene may comprise a dominant negative form of any nicotine biosynthetic pathway transcription factor that positively regulates nicotine biosynthesis, such as, but not limited to, those described herein. The dominant negative form of the aforementioned transcription factors may comprise a translational fusion of at least one DNA binding domain of the transcription factor gene and a transcriptional repressor domain, such as an Engrailed (En) protein transcriptional repressor domain (SEQ ID NO: 11). Transgenic tobacco cells and plants of the present technology are characterized by lower nicotine content than non-transformed control tobacco cells and plants.

Tobacco plants with extremely low levels of nicotine production, or no nicotine production, are attractive as recipients for transgenes expressing commercially valuable products such as pharmaceuticals, cosmetic components, or food additives. Tobacco is attractive as a recipient plant for a transgene encoding desirable product, as tobacco is easily genetically engineered and produces a very large biomass per acre; tobacco plants with reduced resources devoted to nicotine production accordingly will have more resources available for production of transgene products. Methods of transforming tobacco with transgenes producing desired products are known in the art; any suitable technique may be utilized with the low nicotine tobacco plants of the present invention.

Tobacco plants according to the present technology with reduced expression of one or more of the transcription factors described herein and reduced nicotine levels will be desirable in the production of tobacco products having reduced nicotine content. Tobacco plants according to the present technology will be suitable for use in any tobacco product, including but not limited to chewing, pipe, cigar, and cigarette tobacco, snuff, and cigarettes made from the reduced-nicotine tobacco, and may be in any form including leaf tobacco, shredded tobacco, or cut tobacco.

The constructs of the present technology may also be useful in providing transgenic plants having increased expression of one or more of transcription factors that positively regulate the expression of nicotine biosynthetic pathway enzymes and increased nicotine content in the plant. Such constructs, methods using these constructs and the plants so produced may be desirable in the production of tobacco products having altered nicotine content, or in the production of plants having nicotine content increased for its insecticidal effects.

### II. Definitions

All technical terms employed in this specification are commonly used in biochemistry, molecular biology and agriculture; hence, they are understood by those skilled in the field to which the present technology belongs. Those technical terms can be found, for example in: Molecular Cloning: A Laboratory Manual 3rd ed., vol. 1-3, ed. Sambrook and Russel (Cold Spring Harbor Laboratory Press, Cold Spring Harbor, N.Y., 2001); Current Protocols In Molecular Biology, ed. Ausubel et al. (Greene Publishing Associates and Wiley-Interscience, New York, 1988) (including periodic updates); Short Protocols In Molecular Biology: A Compendium Of Methods From Current Protocols In Molecular Biology 5th ed., vol. 1-2, ed. Ausubel et al. (John Wiley & Sons, Inc., 2002); Genome Analysis: A Laboratory Manual, vol. 1-2, ed. Green et al. (Cold Spring Harbor Laboratory Press, Cold Spring Harbor, N.Y., 1997). Methodology involving plant biology techniques are described here and also are described in detail in treatises such as Methods In Plant Molecular Biology: A Laboratory Course Manual, ed. Maliga et al. (Cold Spring Harbor Laboratory Press, Cold Spring Harbor, N.Y., 1995).

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

An “alkaloid” is a nitrogen-containing basic compound found in plants and produced by secondary metabolism. A “pyrrolidine alkaloid” is an alkaloid containing a pyrrolidine ring as part of its molecular structure, for example, nicotine. Nicotine and related alkaloids are also referred to as pyridine alkaloids in the published literature. A “pyridine alkaloid” is an alkaloid containing a pyridine ring as part of its molecular structure, for example, nicotine. A “nicotinic alkaloid” is nicotine or an alkaloid that is structurally related to nicotine and that is synthesized from a compound produced in the nicotine biosynthesis pathway. Illustrative nicotinic alkaloids include but are not limited to nicotine, nornicotine, anatabine, anabasine, anatalline, N-methylanatabine, N-methylanabasine, myosmine, anabaseine, formylnornicotine, nicotyrine, and cotinine. Other very minor nicotinic alkaloids in tobacco leaf are reported, for example, in Hecht et al., Accounts of Chemical Research 12: 92-98 (1979); Tso, T. G., Production, Physiology and Biochemistry of Tobacco Plant. Ideals Inc., Beltsville, Mo. (1990).

As used herein “alkaloid content” means the total amount of alkaloids found in a plant, for example, in terms of pg/g dry weight (DW) or ng/mg fresh weight (FW). “Nicotine content” means the total amount of nicotine found in a plant, for example, in terms of mg/g DW or FW.

The term “biologically active fragment” means a fragment of a nicotine biosynthetic pathway transcription factor, which can, for example, bind to an antibody that will also bind the full length transcription factor. The term “biologically active fragment” can also mean a fragment of nicotine biosynthetic pathway transcription factor which can, for example, encode for a protein or fragment thereof that binds to DNA or that activates transcription either by binding to DNA itself or by interacting with a DNA-binding protein. In some embodiments, a biologically active fragment of nicotine biosynthetic pathway transcription factor can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% of the full length sequence (either amino acid or nucleic acid) as set forth in SEQ ID NOs: 1-10.

A “chimeric nucleic acid” comprises a coding sequence or fragment thereof linked to a nucleotide sequence that is different from the nucleotide sequence with which it is associated in cells in which the coding sequence occurs naturally.

The terms “encoding” and “coding” refer to the process by which a gene, through the mechanisms of transcription and translation, provides information to a cell from which a series of amino acids can be assembled into a specific amino acid sequence to produce an active enzyme. Because of the degeneracy of the genetic code, certain base changes in DNA sequence do not change the amino acid sequence of a protein.

“Endogenous nucleic acid” or “endogenous sequence” is “native” to, i.e., indigenous to, the plant or organism that is to be genetically engineered. It refers to a nucleic acid, gene, polynucleotide, DNA, RNA, mRNA, or cDNA molecule that is present in the genome of a plant or organism that is to be genetically engineered.

“Exogenous nucleic acid” refers to a nucleic acid, DNA or RNA, which has been introduced into a cell (or the cell's ancestor) through the efforts of humans. Such exogenous nucleic acid may be a copy of a sequence which is naturally found in the cell into which it was introduced, or fragments thereof.

As used herein, “expression” denotes the production of an RNA product through transcription of a gene or the production of the polypeptide product encoded by a nucleotide sequence. “Overexpression” or “up-regulation” is used to indicate that expression of a particular gene sequence or variant thereof, in a cell or plant, including all progeny plants derived thereof, has been increased by genetic engineering, relative to a control cell or plant.

“Fusion proteins” or “translational fusions” can be created by the joining of translational sequences from two different genes to create a hybrid or chimeric protein molecule. For example, fusion proteins of the present technology may comprise a transcription repressor domain in translational fusion with a transcription factor or fragment thereof that positively regulates nicotine biosynthesis. Such fusion proteins, comprising a transcription repressor domain, yield dominant negative forms of the transcription factor.

“Genetic engineering” encompasses any methodology for introducing a nucleic acid or specific mutation into a host organism. For example, a plant is genetically engineered when it is transformed with a polynucleotide sequence that suppresses expression of a gene, such that expression of a target gene is reduced compared to a control plant. A plant is genetically engineered when a polynucleotide sequence is introduced that results in the expression of a novel gene in the plant, or an increase in the level of a gene product that is naturally found in the plants. In the present context, “genetically engineered” includes transgenic plants and plant cells, as well as plants and plant cells produced by means of chimeric repressor silencing technology (CRES-T), such as that described by Hiratsu et al., The Plant Journal 34:733-739 (2003).

“Heterologous nucleic acid” refers to a nucleic acid, DNA, or RNA, which has been introduced into a cell (or the cell's ancestor), and which is not a copy of a sequence naturally found in the cell into which it is introduced. Such heterologous nucleic acid may comprise segments that are a copy of a sequence that is naturally found in the cell into which it has been introduced, or fragments thereof.

By “isolated nucleic acid molecule” is intended a nucleic acid molecule, DNA, or RNA, which has been removed from its native environment. For example, recombinant DNA molecules contained in a DNA construct are considered isolated for the purposes of the present technology. Further examples of isolated DNA molecules include recombinant DNA molecules maintained in heterologous host cells or DNA molecules that are purified, partially or substantially, in solution. Isolated RNA molecules include in vitro RNA transcripts of the DNA molecules of the present technology. Isolated nucleic acid molecules, according to the present technology, further include such molecules produced synthetically.

Nicotine is the major alkaloid in Nicotiana tabacum and some other species in the Nicotiana genus. Other plants have nicotine-producing ability, including, for example, Duboisia, Anthoceriscis, and Salpiglossis genera in the Solanaceae, and Eclipta, and Zinnia genera in the Compositae.

“Plant” is a term that encompasses whole plants, plant organs (e.g., leaves, stems, roots, etc.), seeds, differentiated or undifferentiated plant cells, and progeny of the same. Plant material includes without limitation seeds, suspension cultures, embryos, meristematic regions, callus tissues, leaves, roots, shoots, stems, fruit, gametophytes, sporophytes, pollen, and microspores. The class of plants that can be used in the present technology is generally as broad as the class of higher plants amenable to transformation techniques, including both monocotyledonous and dicotyledonous plants. In some embodiments, the plant has a nicotine-producing capacity, such as plants of the Nicotiana, Duoisia, Anthocericis, and Salpiglossis genera in Solanaceae or the Eclipta and Zinnia genera in Compositae. In some embodiments, the plant is Nicotiana tabacum.

“Plant cell culture” means cultures of plant units such as, for example, protoplasts, cell culture cells, cells in plant tissues, pollen, pollen tubes, ovules, embryo sacs, zygotes, and embryos at various stages of development. In some embodiments of the present technology, a transgenic tissue culture or transgenic plant cell culture is provided, wherein the transgenic tissue or cell culture comprises a nucleic acid molecule of the present technology.

“Decreased alkaloid plant” or “reduced alkaloid plant” encompasses a genetically engineered plant that has a decrease in alkaloid content to a level less than 50%, and preferably less than 10%, 5%, or 1% of the alkaloid content of a non-transformed control plant of the same species or variety.

“Decreased nicotine plant” or “reduced nicotine plant” encompasses a genetically engineered plant that has a decrease in nicotine content to a level less than 50%, and preferably less than 10%, 5%, or 1% of the nicotine content of a non-transformed control plant of the same species or variety.

“Increased alkaloid plant” encompasses a genetically engineered plant that has an increase in alkaloid content greater than 10%, and preferably greater than 50%, 100%, or 200% of the alkaloid content of a non-transformed control plant of the same species or variety.

“Increased nicotine plant” encompasses a genetically engineered plant that has an increase in nicotine content greater than 10%, and preferably greater than 50%, 100%, or 200% of the nicotine content of a non-transformed control plant of the same species or variety.

“Loss of function” refers to the loss of function of one or more of the nicotine biosynthetic pathway transcription factor proteins described herein in a host tissue or organism, and encompasses the function at the molecular level (e.g., loss of transcriptional activation of downstream target genes of one or more of the transcription factors described herein), and also at the phenotypic level (e.g., reduced nicotine levels).

“Promoter” connotes a region of DNA upstream from the start of transcription that is involved in recognition and binding of RNA polymerase and other proteins to initiate transcription. A “constitutive promoter” is one that is active throughout the life of the plant and under most environmental conditions. Tissue-specific, tissue-preferred, cell type-specific, and inducible promoters constitute the class of “non-constitutive promoters.” “Operably linked” refers to a functional linkage between a promoter and a second sequence, where the promoter sequence initiates and mediates transcription of the DNA sequence corresponding to the second sequence. In general, “operably linked” means that the nucleic acid sequences being linked are contiguous.

“Sequence identity” or “identity” in the context of two polynucleotide (nucleic acid) or polypeptide sequences includes reference to the residues in the two sequences that are the same when aligned for maximum correspondence over a specified region. When percentage of sequence identity is used in reference to proteins it is recognized that residue positions which are not identical often differ by conservative amino acid substitutions, where amino acid residues are substituted for other amino acid residues with similar chemical properties, such as charge and hydrophobicity, and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, for example, according to the algorithm of Meyers & Miller, Computer Applic. Biol. Sci. 4: 11-17 (1988), as implemented in the program PC/GENE (Intelligenetics, Mountain View, Calif., USA).

Use in this description of a percentage of sequence identity denotes a value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The terms “suppression” or “down-regulation” are used synonymously to indicate that expression of a particular gene sequence variant thereof, in a cell or plant, including all progeny plants derived thereof, has been reduced by genetic engineering, relative to a control cell or plant.

As used herein, a “synergistic effect” refers to a greater-than-additive effect which is produced by a combination of at least two compounds (e.g., the effect produced by a combined overexpression of at least two dominant negative transcription factors), and which exceeds that which would otherwise result from the individual compound (e.g., the effect produced by the overexpression of a single dominant negative transcription factor alone).

“Tobacco” or “tobacco plant” refers to any species in the Nicotiana genus that produces nicotinic alkaloids, including but not limited to the following: Nicotiana acaulis, Nicotiana acuminata, Nicotiana acuminata var. multzjlora, Nicotiana africana, Nicotiana alata, Nicotiana amplexicaulis, Nicotiana arentsii, Nicotiana attenuata, Nicotiana benavidesii, Nicotiana benthamiana, Nicotiana bigelovii, Nicotiana bonariensis, Nicotiana cavicola, Nicotiana clevelandii, Nicotiana cordifolia, Nicotiana corymbosa, Nicotiana debneyi, Nicotiana excelsior, Nicotiana forgetiana, Nicotiana fragrans, Nicotiana glauca, Nicotiana glutinosa, Nicotiana goodspeedii, Nicotiana gossei, Nicotiana hybrid, Nicotiana ingulba, Nicotiana kawakamii, Nicotiana knightiana, Nicotiana langsdorfi, Nicotiana linearis, Nicotiana longiflora, Nicotiana maritima, Nicotiana megalosiphon, Nicotiana miersii, Nicotiana noctiflora, Nicotiana nudicaulis, Nicotiana obtusifolia, Nicotiana occidentalis, Nicotiana occidentalis subsp. hesperis, Nicotiana otophora, Nicotiana paniculata, Nicotiana pauczjlora, Nicotiana petunioides, Nicotiana plumbaginifolia, Nicotiana quadrivalvis, Nicotiana raimondii, Nicotiana repanda, Nicotiana rosulata, Nicotiana rosulata subsp. ingulba, Nicotiana rotundifolia, Nicotiana rustica, Nicotiana setchellii, Nicotiana simulans, Nicotiana solanifolia, Nicotiana spegauinii, Nicotiana stocktonii, Nicotiana suaveolens, Nicotiana sylvestris, Nicotiana tabacum, Nicotiana thyrsiflora, Nicotiana tomentosa, Nicotiana tomentosifomis, Nicotiana trigonophylla, Nicotiana umbratica, Nicotiana undulata, Nicotiana velutina, Nicotiana wigandioides, and interspecific hybrids of the above.

“Tobacco product” refers to a product comprising material produced by a Nicotiana plant, including for example, cut tobacco, shredded tobacco, nicotine gum and patches for smoking cessation, cigarette tobacco including expanded (puffed) and reconstituted tobacco, cigar tobacco, pipe tobacco, cigarettes, cigars, and all forms of smokeless tobacco such as chewing tobacco, snuff, snus, and lozenges.

Tobacco-specific nitrosamines (TSNAs) are a class of carcinogens that are predominantly formed in tobacco during curing, processing, and smoking. Hoffman, D., et al., J. Natl. Cancer Inst. 58:1841-4 (1977); Wiernik A et al., Recent Adv. Tob. Sci., 21:39-80 (1995). TSNAs, such as 4-(N-nitrosomethylamino)-1-(3-pyridyl)-1-butanone (NNK), N′-nitrosonornicotine (NNN), N′-nitrosoanatabine (NAT), and N′-nitrosoanabasine (NAB), are formed by N-nitrosation of nicotine and other minor Nicotiana alkaloids, such as nornicotine, anatabine, and anabasine. Reducing nicotinic alkaloids reduces the level of TSNAs in tobacco and tobacco products.

As used herein, “transformation” refers to the introduction of exogenous nucleic acid into cells, so as to produce transgenic cells stably transformed with the exogenous nucleic acid.

A “transcription factor” is a protein that binds that binds to DNA regions, typically promoter regions, using DNA binding domains and increases or decreases the transcription of specific genes. A transcription factor “positively regulates” alkaloid biosynthesis if expression of the transcription factor increases the transcription of one or more genes encoding alkaloid biosynthesis enzymes and increases alkaloid production. A transcription factor “negatively regulates” alkaloid biosynthesis if expression of the transcription factor decreases the transcription of one or more genes encoding alkaloid biosynthesis enzymes and decreases alkaloid production. Transcription factors are classified based on the similarity of their DNA binding domains. (See, e.g., Stegmaier et al., Genome Inform. 15 (2): 276-86 ((2004)). Classes of plant transcription factors include ERF transcription factors; Myc basic helix-loop-helix transcription factors; homeodomain leucine zipper transcription factors; AP2 ethylene-response factor transcription factors; and B3 domain, auxin response factor transcription factors.

A “variant” is a nucleotide or amino acid sequence that deviates from the standard, or given, nucleotide or amino acid sequence of a particular gene or polypeptide. The terms “isoform,” “isotype,” and “analog” also refer to “variant” forms of a nucleotide or an amino acid sequence. An amino acid sequence that is altered by the addition, removal, or substitution of one or more amino acids, or a change in nucleotide sequence, may be considered a variant sequence. A polypeptide variant may have “conservative” changes, wherein a substituted amino acid has similar structural or chemical properties, e.g., replacement of leucine with isoleucine. A polypeptide variant may have “nonconservative” changes, e.g., replacement of a glycine with a tryptophan. Analogous minor variations may also include amino acid deletions or insertions, or both. Guidance in determining which amino acid residues may be substituted, inserted, or deleted may be found using computer programs well known in the art such as Vector NTI Suite (InforMax, MD) software. Variant may also refer to a “shuffled gene” such as those described in Maxygen-assigned patents (see, e.g., U.S. Pat. No. 6,602,986).

### III. Genetic Engineering of Plants and Cells Using Dominant Negative Forms of Transcription Factors Capable of Reducing Expression of Endogenous Nicotine Biosynthesis Genes

The present technology contemplates genetically engineering loss-of-function phenotypes (of host cells, tissues, or plants and portions thereof) by expressing a nucleic acid sequence encoding a protein fusion of a nicotine biosynthetic pathway transcription factor protein (Table 1) with a (dominant) repressor domain. In particular, the present technology contemplates the use of dominant negative forms of a nicotine biosynthetic pathway transcription factor protein comprising a chimeric nucleic acid construct encoding translational fusion of at least one nicotine biosynthetic pathway transcription factor or biologically active fragment thereof to any transcriptional repressor domain that functions in plant cells, including but not limited to an Engrailed (En) repressor domain (e.g., En²⁹⁸; SEQ ID NO: 11) or an EAR-motif repressor domain. Also included are the NtERF3 and AtERF4 genes, which belong to the class-II ethylene-responsive element-binding factor (ERF) genes, and SUPERMAN (SUP) (SEQ ID NO: 16), each of which is characterized by a conserved ERF-associated amphiphilic repression (EAR) motif that has been functionally identified as a repression domain. See Ohta et al., Plant Cell 13:1959-1968 (2001). Studies have shown that the repressor domains of the class-II ERF and TFIIIA-type zinc finger repressors of transcription that include SUPERMAN (SUP) contain the EAR motif, and when short peptides that contained the EAR motif were fused to activators of transcription, the resultant chimeric transcription factors acted as strong repressors and suppressed the expression of a reporter gene in the presence of another activator of transcription in expression assays in Arabidopsis. (See Hiratsu et al., FEBS Lett. 514:351-354 (2002); Ohta et al. (2001)). Additional repressor domains suitable for use in the present technology include a 12-amino acid sequence of the AtERF4 transcription factor (SRDX; SEQ ID NO: 12) and a 24-amino acid sequence of the AtERF4 transcription factor (SEQ ID NO: 13). Other plant repressors contemplated by the present technology include LEUNIG (SEQ ID NO: 18), and SEUSS (SEQ ID NO: 19). In addition, the present technology contemplates the use of animal repressors with transferable repression domains, which have been identified in a number of proteins, including WT1, eve, c-ErbA, and v-ErbA. See Hanna-Rose & Hansen, Trends Genet., 12:229-234 (1996). Illustrative, non-limiting examples of repressor domains suitable for use in the present technology are provided in Table 2.

TABLE 2
Repressor Domains for the Construction of Chimeric Repressors

| Repressor | | | |
|---|---|---|---|
| Domain/Protein | | | |
| Containing | | | |
| Repressor Domain | Sequence | Function | Source |
| ENGRAILED | SEQ ID NO: 11 | Active | U.S. Patent |
| (En²⁹⁸) | | transcriptional | Application |
| (N-terminal amino | | repressor | Publication |
| acids 1-298 of the | | | 2008/0196124 |
| Engrailed | | | |
| homeodomain | | | |
| protein from | | | |
| Drosophila) | | | |
| SRDX | LDLDLELRLGFA (SEQ ID NO: 12) | EAR-motif | Hiratsu et al., |
| (A 12-amino acid | | repressor | Plant 1, 34: 733- |
| sequence of the | | domain | 739 (2003). |
| Arabidopsis | | | |
| ethylene-responsive | | | |
| transcription factor 4 | | | |
| (AtERF4)) | | | |
| AtERF4 | EGGMEKRSQLLDLDLNLPPPSEQA | Ethylene- | Chandler & Werr, |
| (A 24-amino acid | (SEQ ID NO: 13) | responsive | TRENDS in Plant |
| sequence of the | | element | Science, 8(6): 279- |
| Arabidopsis | | binding factor, | 285 (2003). |
| ethylene-responsive | | characterized | |
| transcription factor 4 | | by an EAR- | |
| (AtERF4)) | | motif repressor | |
| | | domain | |
| NtERF3 | VGPTVSDSSSAVEENQYDGKRGID | Ethylene- | Ohta et al., Plant |
| (191-225 aa) | LDLNLAPPMEF | responsive | Cell, 13(8): 1959- |
| | (SEQ ID NO: 15) | element | 1968 (2001). |
| | | binding factor, | |
| | | characterized | |
| | | by an EAR- | |
| | | motif repressor | |
| | | domain | |
| SUPERMAN | NDEIISLELEIGLINESEQDLDL | EAR-motif | Hiratsu et al., |
| (175-204 aa) | ELRLGFA | repressor | FEBS Lett., |
| | (SEQ ID NO: 17) | domain | 514: 351-354 |
| | | | (2002). |
| LEUNIG | SEQ ID NO: 18 | Plant repressor. | Mayer et al., |
| | | | Nature, |
| | | | 402(6763): 769- |
| | | | 777 (1999). |
| SEUSS | SEQ ID NO: 19 | Plant repressor. | Franks et al., |
| | | | Development, |
| | | | 129(1): 253-263 |
| | | | (2002). |

As an example, to provide loss-of-function and produce a dominant negative effect, nicotine biosynthetic pathway transcription factor protein fusions are made with a 12-amino acid “EAR” repressor domain such as SRDX (SEQ ID NO: 12) as described by Hiratsu et al. (Plant J., 34:733-739 (2003)). These repressor domain fusions to any one of the nicotine biosynthetic pathway transcription factor genes are able to cause repression of downstream target genes and thus result in an effective loss-of-function mutant (dominant negative effect). These repressor fusions also effect repression in heterologous plants wherein the orthologous genes have not yet been identified. In some embodiments, the nucleic acid constructs further comprise corepressor sequences associated with the repressor sequence. The corepressors may include but are not limited to KAP-1, groucho, KOX-1, N-Cor, or SMRT.

In some embodiments, a nucleic acid sequence is provided that encodes a chimeric repressor domain-nicotine biosynthesis transcription factor protein fusion protein, such as an NtERF32-SRDX fusion protein. In addition, a vector comprising the nucleic acid sequence and a host cell, tissue, and/or organisms comprising the chimeric gene are provided. To generate a nicotine biosynthetic pathway transcription factor-repressor domain fusion protein, the nucleic acid sequence encoding the repressor domain is translationally fused to the nucleic acid sequence comprising the transcription factor coding sequence.

The transcription factor-repressor domain fusion protein encoding nucleic acid sequence (e.g., NtERF32-SRDX) can be placed under the control of a constitutive or specific promoter (e.g., tissue specific or developmentally regulated) that is operably linked to a polyadenylation or terminator region. Constitutive expression provides a loss-of-function in all host tissues where the nicotine biosynthetic pathway transcription factor is expressed and required for function. Specific expression of the fusion protein provides a loss-of-function in the specific tissue or under a particular condition.

A variety of promoters can be used in the practice of the present technology. Exemplary, non-limiting promoters include a viral promoter such as a CaMV35S or FMV35S promoter. The CaMV35S and FMV35S promoters are active in a variety of transformed plant tissues and most plant organs (e.g., callus, leaf, seed, and root). Enhanced or duplicate versions of CaMV35S and FMV35S promoters are particularly useful in the practice of the present technology. Other promoters useful in the present technology include nopaline synthase (NOS) and octopine synthase (OCS) promoters, the cauliflower mosaic virus (CaMV) 19S promoters, the light-inducible promoter from the small subunit of ribulose 1,5-bisphosphate carboxylase (ssRUBSICO), the rice Act1 promoter, and the Figwort Mosaic Virus (FMV) 35S promoter.

It is also anticipated that root specific promoters can be particularly useful for driving expression of nicotine biosynthetic pathway transcription factors since nicotine is produced in the roots of tobacco plants. In some embodiments, the TobRD2 root-cortex specific promoter may be utilized. (See, e.g., U.S. Pat. No. 5,837,876).

Another illustrative example of a dominant negative nicotine biosynthetic pathway transcription factor form that can be used in the practice of the present technology comprises a nucleic acid encoding translational fusion of, for example, the NtERF32 gene to an Engrailed (En²⁹⁸) protein transcriptional repressor domain to form an NtERF32-En²⁹⁸ fusion protein. Engrailed fusions have been shown to provide for dominant-negative transgenes that phenocopy the effects of loss-of-function mutations in those transgenes (see Markel et al., Nucleic Acids Research, 30(21): 4709-4719 (2002)). The first 298 amino acids (SEQ ID NO: 11) encoded by the Engrailed gene (En²⁹⁸) or any other repressor domain may provide for transcriptional repression when fused to the N-terminus or C-terminus of complete or truncated nicotine biosynthetic pathway transcription factors that include at least one DNA binding domain. Such fusions can be effected by in-frame fusions of the DNA fragment encoding the first 298 amino acids of the Engrailed protein to a second DNA fragment encoding the N-terminus or C-terminus of a complete or truncated nicotine biosynthetic pathway transcription factor or biologically active fragment thereof.

Another illustrative example of a dominant negative nicotine biosynthetic pathway transcription factor-repressor domain fusion of the present technology can be generated by fusing the following 12 specific amino acids (LDLDLELRLGFA; “SRDX”; SEQ ID NO: 12), for example, in frame to the C-terminal of, for example, an NtERF32 protein (SEQ ID NO: 4) to form an NtERF32-EAR (e.g., NtERF32-SRDX) fusion protein. To generate an illustrative nicotine biosynthetic pathway transcription factor-repressor domain fusion protein of the present technology, the EAR domain encoding nucleic acid sequences, such as SEQ ID NO: 20, may be added in frame to the 3′ end of the NtERF32 coding sequence, followed by a stop codon (e.g., TAA).

EAR Repressor Coding Sequence:

(SEQ ID NO: 20)
5′ - CTG GAT CTG GAT CTA GAA CTC CGT TTG GGT TTC
GCT (TAA) -3′

Similar experiments with fusions of the EAR repressor domain to other plant transcription factors have shown that EAR fusions provide for dominant-negative transgenes that dominantly repress expression of the genes they ordinarily regulate (see Hiratsu et al., Plant J., 34(5):733-739 (2003)). The EAR repressor domain comprising either the 12-amino acid sequence (SEQ ID NO: 12) or 24-amino acid sequence (EGGMEKRSQLLDLDLNLPPPSEQA; SEQ ID NO: 13) of the Arabidopsis Ethylene-responsive transcription factor 4 (ERF4; Tiwari et al., The Plant Cell, 16:533-543 (2004)) may provide for transcriptional repression when fused to either the N-terminus or C-terminus of a complete or truncated plant transcription factor or biologically active fragment thereof.

The particular nicotine biosynthetic pathway transcription factor-repressor domain fusion protein and the number of different fusion proteins will vary depending upon the degree of inhibition desired. In some embodiments, one or more nicotine biosynthetic pathway transcription factors are selected for fusion with a repressor domain and concurrent expression in a transformed cell or plant. One of skill in the art will be guided in the selection of the appropriate combination of dominant repressor forms of the nicotine biosynthetic pathway transcription factors disclosed herein using techniques known in the art to achieve the desired nicotine content when transformed into a recipient plant cell or plant.

A. Quantifying Nicotine Content

Methods of ascertaining nicotine content are available to those skilled in the art. In some embodiments of the present technology, genetically engineered plants and cells are characterized by reduced nicotine content.

A quantitative reduction in nicotine levels can be assayed by several methods, as for example by quantification based on gas-liquid chromatography, high performance liquid chromatography, radio-immunoassays, and enzyme-linked immunosorbent assays.

In describing a plant of the present technology, the phrase “decreased nicotine plant” or “reduced nicotine plant” encompasses a plant that has a decrease in nicotine content to a level less than about 50%, about 40%, about 30%, about 25%, about 20%, about 15%, about 10%, about 9%, about 8%, about 7%, about 6%, about 5%, about 4%, about 3%, about 2% or about 1% of the nicotine content of a control plant of the same species or variety.

B. Nicotine Biosynthetic Pathway Transcription Factor Sequences

Transcription factors implicated in the positive regulation of nicotine biosynthesis have been identified in several plants. Accordingly, the present technology contemplates any nucleic acid, gene, polynucleotide, DNA, RNA, mRNA, or cDNA molecule that is isolated from the genome of a plant species that up-regulates nicotinic alkaloid biosynthesis. Examples of such transcription factor open reading frames (ORFs) or genes include the sequences set forth in SEQ ID NOs: 1-10, including biologically active fragments thereof.

The present technology also includes “variants” of SEQ ID NOs: 1-10, with one or more bases deleted, substituted, inserted, or added, which variant codes for a polypeptide that regulates alkaloid biosynthesis activity. Accordingly, sequences having “base sequences with one or more bases deleted, substituted, inserted, or added” retain physiological activity even when the encoded amino acid sequence has one or more amino acids substituted, deleted, inserted, or added. Additionally, multiple forms of the transcription factors of the present technology may exist, which may be due to post-translational modification of a gene product, or to multiple forms of the transcription factor gene. Nucleotide sequences that have such modifications and that code for a transcription factor that positively regulates alkaloid biosynthesis are included within the scope of the present technology.

A transcription factor sequence can be synthesized ab initio from the appropriate bases, for example, by using an appropriate protein sequence disclosed herein as a guide to create a DNA molecule that, though different from the native DNA sequence, results in the production of a protein with the same or similar amino acid sequence.

Unless otherwise indicated, all nucleotide sequences determined by sequencing a DNA molecule herein were determined using an automated DNA sequencer, such as the Model 3730xl from Applied Biosystems, Inc. Therefore, as is known in the art for any DNA sequence determined by this automated approach, any nucleotide sequence determined herein may contain some errors. Nucleotide sequences determined by automation are typically at least about 95% identical, more typically at least about 96% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence may be completely different from the amino acid sequence actually encoded by the sequenced DNA molecule, beginning at the point of such an insertion or deletion.

For purposes of the present technology, two sequences hybridize under stringent conditions when they form a double-stranded complex in a hybridization solution of 6×SSE, 0.5% SDS, 5×Denhardt's solution and 100 μg of non-specific carrier DNA. See Ausubel, et al., supra, at section 2.9, supplement 27 (1994). Sequences may hybridize at “moderate stringency,” which is defined as a temperature of 60° C. in a hybridization solution of 6×SSE, 0.5% SDS, 5×Denhardt's solution and 100 μg of non-specific carrier DNA. For “high stringency” hybridization, the temperature is increased to 68° C. Following the moderate stringency hybridization reaction, the nucleotides are washed in a solution of 2×SSE plus 0.05% SDS for five times at room temperature, with subsequent washes with 0.1×SSC plus 0.1% SOS at 60° C. for 1 h. For high stringency, the wash temperature is increased to 68° C. For the purpose of the technology, hybridized nucleotides are those that are detected using 1 ng of a radiolabeled probe having a specific radioactivity of 10,000 cpm/ng, where the hybridized nucleotides are clearly visible following exposure to X-ray film at −70° C. for no more than 72 hours.

The present technology encompasses nucleic acid molecules which are at least about 90%, about 91%, about 92%, about 93%, about 94%, about 95%, about 96%, about 97%, about 98%, about 99% or 100% identical to a nucleic acid sequence described in any of SEQ ID NOs: 1-10. Differences between two nucleic acid sequences may occur at the 5′ or 3′ terminal positions of the reference nucleotide sequence or anywhere between those terminal positions, interspersed either individually among nucleotides in the reference sequence or in one or more contiguous groups within the reference sequence.

C. Host Plants and Cells

In some embodiments, the present technology relates to the genetic manipulation of a plant or cell via introducing a chimeric nucleic acid sequence that encodes a fusion protein comprising a nicotine biosynthetic pathway transcription factor and a repressor domain. In some embodiments, expression of such fusion proteins produces a dominant negative effect resulting in reduced nicotine biosynthesis in the plant or cell. Accordingly, the present technology provides methodology and constructs for reducing nicotine biosynthesis in a plant. Additionally, the present technology provides methods for reducing nicotine biosynthesis in a plant cell.

Plants for use in the methods of the present technology are species of Nicotiana, or tobacco, including N. tabacum, N. rustica, and N. glutinosa. Any strain or variety of tobacco may be used. In some embodiments, strains that are already low in nicotine content, such as Nic1/Nic2 double mutants, are used in the methods of the present technology.

Any plant tissue capable of subsequent clonal propagation, whether by organogenesis or embryogenesis, may be transformed with a vector of the present technology. The term “organogenesis,” as used herein, means a process by which shoots and roots are developed sequentially from meristematic centers; the term “embryogenesis,” as used herein, means a process by which shoots and roots develop together in a concerted fashion (not sequentially), whether from somatic cells or gametes. The particular tissue chosen will vary depending on the clonal propagation systems available for, and best suited to, the particular species being transformed. Exemplary tissue targets include leaf disks, pollen, embryos, cotyledons, hypocotyls, callus tissue, existing meristematic tissue (e.g., apical meristems, axillary buds, and root meristems), and induced meristem tissue (e.g., cotyledon meristem and hypocotyl meristem).

Plants of the present technology may take a variety of forms. The plants may be chimeras of transformed cells and non-transformed cells; the plants may be clonal transformants (e.g., all cells transformed to contain the transcription cassette); the plants may comprise grafts of transformed and untransformed tissues (e.g., a transformed root stock grafted to an untransformed scion in citrus species). The transformed plants may be propagated by a variety of means, such as by clonal propagation or classical breeding techniques. For example, first generation (or T1) transformed plants may be selfed to give homozygous second generation (or T2) transformed plants, and the T2 plants further propagated through classical breeding techniques. A dominant selectable marker (such as npill) can be associated with the transcription cassette to assist in breeding.

In view of the foregoing, it will be apparent that plants which may be employed in practicing the present invention include those of the genus Nicotiana.

Methods of making recombinant plants of the present technology, in general, involve first providing a plant cell capable of regeneration. The plant cell is then transformed with a DNA construct comprising a transcription cassette of the present technology and a recombinant plant is regenerated from the transformed plant cell. Any of the transgenes used for reducing the expression of an endogenous nicotine biosynthetic pathway transcription factor gene can be introduced into the genome of a host plant via techniques known in the art such as Agrobaterium-mediated transformation, Rhizobium-mediated transformation, Sinorhizobium-mediated transformation, particle-mediated transformation (e.g., bombarding the plant cell with microparticles carrying the transcription cassette), DNA transfection, DNA electroporation, or any other technique suitable for the production of a transgenic plant. The aforementioned methods of introducing transgenes are well known to those skilled in the art and any of the methods can be used to produce a tobacco plant having decreased expression of nicotine biosynthetic pathway transcription factors, and thus lower nicotine content than a non-transformed control tobacco plant.

Numerous Agrobacterium vector systems useful in methods of the present technology are known. For example, U.S. Pat. No. 4,459,355 discloses a method for transforming susceptible plants, including dicots, with an Agrobacterium strain containing the Ti plasmid. The transformation of woody plants with an Agrobacterium vector is disclosed in U.S. Pat. No. 4,795,855. Further, U.S. Pat. No. 4,940,838 discloses a binary Agrobacterium vector (i.e., one in which the Agrobacterium contains one plasmid having the vir region of a Ti plasmid but no T region, and a second plasmid having a T region but no vir region) useful in carrying out the present invention.

Microparticles comprising a DNA construct of the present invention, which microparticle is suitable for the ballistic transformation of a plant cell, are also useful for making transformed plants of the present technology. The microparticle is propelled into a plant cell to produce a transformed plant cell, and a plant is regenerated from the transformed plant cell. Any suitable ballistic cell transformation methodology and apparatus can be used in practicing the methods of the present technology. Exemplary apparatus and procedures are disclosed in U.S. Pat. Nos. 4,945,050 and 5,015,580. When using ballistic transformation procedures, the transcription cassette may be incorporated into a plasmid capable of replicating in or integrating into the cell to be transformed. Examples of microparticles suitable for use in such systems include about 1 to about 5 μm gold spheres. The DNA construct may be deposited on the microparticle by any suitable technique, such as by precipitation.

Plant species may be transformed with the DNA construct of the present invention by the DNA-mediated transformation of plant cell protoplasts and subsequent regeneration of the plant from the transformed protoplasts in accordance with procedures well known in the art. Fusion of tobacco protoplasts with DNA-containing liposomes or via electroporation is known in the art.

After transformation of the plant cells or plant, those plant cells or plants into which the desired DNA has been incorporated may be selected by such methods as antibiotic resistance, herbicide resistance, tolerance to amino-acid analogues or using phenotypic markers. Transgenic plants are typically obtained by linking the gene of interest (e.g., a transgene capable of reducing expression of a nicotine biosynthetic pathway transcription factor gene) to a selectable marker gene, introducing the linked transgenes into a plant cell by any one of the methods described above, and regenerating the transgenic plant under conditions requiring expression of the selectable marker gene for plant growth. Suitable selectable markers for use in tobacco include, inter alia, antibiotic resistance genes encoding a neomycin phosphotransferase protein (NPTII), a hygromycin phosphotransferase protein (HPT), and chloramphenicol acetyl transferase protein (CAT). Another well-known selectable maker suitable for use in tobacco is a mutant dihydrofolate reductase protein. DNA vectors containing suitable antibiotic resistance genes, and the corresponding antibiotics, are commercially available.

Transformed tobacco cells are selected out of the surrounding population of non-transformed cells by placing the mixed population of cells into a culture medium containing an appropriate concentration of the antibiotic (or other compound normally toxic to tobacco cells) against which the chosen dominant selectable maker gene product confers resistance. Thus, only those tobacco cells that have been transformed will survive and multiply.

Transformed cells are induced to regenerate intact tobacco plants through application of tobacco cell and tissue culture techniques that are well known in the art. The method of plant regeneration is chosen so as to be compatible with the method of transformation. After regeneration of transgenic tobacco plants from transformed cells, the introduced DNA sequence is readily transferred to other tobacco varieties through conventional plant breeding practices and without undue experimentation.

For example, to analyze the segregation of the transgene, regenerated transformed plants (R₀) may be grown to maturity, tested for nicotine levels, and selfed to produce R₁ plants. A percentage of R₁ plants carrying the transgene are homozygous for the transgene. To identify homozygous R₁ plants, transgenic R₁ plants are grown to maturity and selfed. Homozygous RI₁, plants will produce R₂ progeny where each progeny plant carries the transgene; progeny of heterozygous RI₁, plants will segregate 3:1.

As nicotine serves as a natural pesticide which helps protect tobacco plants from damage by pests, it may be desirable to additionally transform low or no nicotine plants produced by the present methods with a transgene (such as Bacillus thuringiensis) that will confer additional insect protection.

Various assays may be used to determine whether the plant cell shows a change in gene expression, for example, Northern blotting or quantitative reverse transcriptase PCR (RT-PCR). Whole transgenic plants may be regenerated from the transformed cell by conventional methods. Such transgenic plants may be propagated and self-pollinated to produce homozygous lines. Such plants produce seeds containing the genes for the introduced trait and can be grown to produce plants that will produce the selected phenotype.

Modified nicotine content, effected in accordance with the present technology, can be combined with other traits of interest, such as disease resistance, pest resistance, high yield or other traits. For example, a stable genetically engineered transformant that contains a suitable transgene that modifies nicotine content may be employed to introgress a modified nicotine content trait into a desirable commercially acceptable genetic background, thereby obtaining a cultivar or variety that combines a modified nicotine level with said desirable background. For example, a genetically engineered tobacco plant with reduced nicotine may be employed to introgress the reduced nicotine trait into a tobacco cultivar with disease resistance trait, such as resistance to TMV, blank shank, or blue mold. Alternatively, cells of a modified alkaloid content plant of the present technology may be transformed with nucleic acid constructs conferring other traits of interest.

D. Reduced-Nicotine Products

The methods of the present technology provide transgenic cells and plants having reduced nicotine levels. For example, the present technology contemplates reducing nicotine levels by expressing dominant negative forms of transcription factors that otherwise positively regulate nicotine biosynthesis to produce a loss-of-function phenotype in the transformed cell or plant.

As described above, tobacco plants with extremely low levels of nicotine production, or no nicotine production, are attractive as recipients for transgenes expressing commercially valuable products such as pharmaceuticals, cosmetic components, or food additives. Tobacco is attractive as a recipient plant for a transgene encoding desirable product, as tobacco is easily genetically engineered and produces a very large biomass per acre; tobacco plants with reduced resources devoted to nicotine production accordingly will have more resources available for production of transgene products.

Tobacco plants according to the present technology with reduced expression of one or more of the transcription factors described herein and reduced nicotine levels will be desirable in the production of tobacco products having reduced nicotine content. Tobacco plants according to the present technology will be suitable for use in any tobacco product, including but not limited to chewing, pipe, cigar, and cigarette tobacco, snuff, and cigarettes made from the reduced-nicotine tobacco for use in smoking cessation, and may be in any form including leaf tobacco, shredded tobacco, or cut tobacco.

Because the present technology provides methods for reducing nicotinic alkaloids, tobacco-specific nitrosamines (TSNAs) may also be reduced as there is a significant, positive correlation between alkaloid content in tobacco and TSNA accumulation. For example, a significant correlation coefficient between anatabine and NAT was 0.76. See Djordjevic et al., J. Agric. Food Chem., 37:752-756 (1989). TSNAs are a class of carcinogens that are predominantly formed in tobacco during curing, processing, and smoking.

TSNAs are considered to be among the most prominent carcinogens in cigarette smoke and their carcinogenic properties are well documented. See Hecht, S. Mutat. Res., 424:127-42 (1999); Hecht, S. Toxicol., 11:559-603 (1998); Hecht et al., Cancer Surv., 8:273-294 (1989). TSNAs have been cited as causes of oral cancer, esophageal cancer, pancreatic cancer, and lung cancer (Hecht & Hoffman, IARC Sci. Publ., 54-61 (1991)). In particular, TSNAs have been implicated as the causative agent in the dramatic rise of adenocarcinoma associated with cigarette smoking and lung cancer (Hoffmann et al., Crit. Rev. Toxicol., 26:199-211 (1996)).

The four TSNAs considered to be the most important by levels of exposure and carcinogenic potency and reported to be possibly carcinogenic to humans are N′-nitrosonornicotine (NNN), 4-methylnitrosoamino-1-(3-pyridyl)-1-butanone (NNK), N′-nitrosoanatabine (NAT) and N′-nitrosoanabasine (NAB) (reviewed in IARC monographs on the evaluation of the carcinogenic risk of chemical to humans Lyon (France) Vol. 37, pp. 205-208 (1985)). These TSNAs are formed by N-nitrosation of nicotine and of the minor Nicotiana alkaloids that include nornicotine, anatabine, and anabasine.

The following levels of alkaloid compounds have been reported for mainstream smoke of non-filter cigarettes (measured in μg/cigarette): nicotine: 100-3000, nornicotine: 5-150, anatabine: 5-15, Anabasine: 5-12 (Hoffmann et al., Chem. Res. Toxicol., 14:7:767-790 (2000)). Mainstream smoke of U.S. cigarettes, with or without filter tips, contain (measured in ng/cigarette): 9-180 ng NNK, 50-500 ng NNN, 3-25 ng NAB and 55-300 ng NAT. Hoffmann et al., J. Toxicol. Environ. Health, 41:1-52 (1994). It is important to note that the levels of these TSNAs in sidestream smoke are 5-10 fold above those in mainstream smoke. Hoffmann et al (1994).

Xie et al. (2004) reported that Vector 21-41, which is a genetically-engineered reduced-nicotine tobacco by the down-regulation of QPT, has a total alkaloid level of about 2300 ppm, which is less than 10 percent of the wild-type tobacco. Mainstream smoke from the Vector 21-41 cigarettes had less than 10 percent of NNN, NAT, NAB, and NNK compared to such levels of a standard full flavor cigarette produced from wild-type tobacco.

The strategy for reducing TSNAs by reducing the corresponding tobacco alkaloid precursors is currently the main focus of agricultural tobacco research. Siminszky et al., Proc. Nat. Acad. Sci. USA, 102(41) 14919-14924 (2005). Thus, to reduce formation of all TSNAs there is an urgent need to reduce the precursor nicotinic alkaloids as much as possible by genetic engineering.

Among others, U.S. Pat. Nos. 5,803,081, 6,135,121, 6,805,134, 6,907,887 and 6,959,712, and U.S. Patent Application Publication Nos. 2005/0034365 and 2005/0072047, discuss methods to reduce TSNAs.

Reduced-nicotine tobacco may also be used to produce reconstituted tobacco (Recon). Recon is produced from tobacco stems and/or smaller leaf particles by a process that closely resembles typical paper making. This process entails processing the various tobacco portions that are to be made into Recon and cutting the tobacco into a size and shape that resembles cut rag tobacco made from whole leaf tobacco. This cut recon then gets mixed with cut-rag tobacco and is ready for cigarette making.

In addition to traditional tobacco products, such as cigarette and cigar tobacco, reduced-nicotine tobacco can be used as a source for protein, fiber, ethanol, and animal feeds. See U.S. Patent Application Publication No. 2002/0197688. For example, reduced-nicotine tobacco may be used as a source of Rubisco (ribulose bisphosphate carboxylase-oxygenase or fraction 1 protein) because unlike other plants, tobacco-derived Rubisco can be readily extracted in crystalline form. With the exception of slightly lower levels of methionine, Rubisco's content of essential amino acids equals or exceeds that of the FAO Provisional Pattern. Ershoff et al., Society for Experimental Biology and Medicine, 157:626-630 (1978); Wildman, S. G. Photosynthesis Research, 73:243-250 (2002)).

For biofuels to replace a sizable portion of the world's dependence on non-renewable energy sources, co-products, such as Rubisco, are required to help defray the cost of producing this renewable energy. Greene et al. Growing Energy. How Biofuels Can End America's Oil Dependence; National Resources Defense Counsel (2004). Thus, the greater reduction in nicotinic alkaloids in tobacco, the greater the likelihood of a successful tobacco biomass system.

### EXAMPLES

The following examples are provided by way of illustration only and not by way of limitation. Those of skill in the art will readily recognize a variety of non-critical parameters that could be changed or modified to yield essentially the same or similar results. The examples should in no way be construed as limiting the scope of the present technology, as defined by the appended claims.

### Example 1: Expression of Chimeric Nicotine Biosynthetic Pathway Transcription Factor-Transcriptional Repressor Fusion Proteins Reduces Nicotine Content in Tobacco BY-2 Cells

This example demonstrates the use of dominant negative forms of nicotine biosynthetic pathway transcription factors to reduce nicotine biosynthesis in plant cell cultures.

### Methods

While tobacco BY-2 cell cultures do not normally synthesize nicotinic alkaloids, methyl jasmonate treatment induces expression of genes for known enzymes in the nicotine biosynthesis pathway and elicits formation of nicotinic alkaloids.

The NtERF32 transcription factor is a positive regulator of nicotine biosynthesis. Transgenic cells expressing the chimeric protein comprising NtERF32 and a transcription repressor domain, such as SRDX fused in frame with the 35S promoter of cauliflower mosaic virus (CaMV) to yield, for example, 35S::NtERF32SRDX, are anticipated to result in the production of reduced levels of nicotine when compared to non-transformed control cells grown under similar conditions.

Cloning and Transformation.

The protein-coding region of the NtERF32 transcription factor gene is amplified by PCR from a Nicotiana tabacum cDNA library with the appropriate 5′ and 3′ primers according to standard methods known in the art. Nucleic acid fragments that correspond to SRDX (LDLDLELRLGFA) (SEQ ID NO: 12) are synthesized with a TAA stop codon at the 3′ end. The resultant nucleic acids are cloned into a transformation vector, such as a Ti plasmid vector, with a promoter, such as the CaMV 35S promoter, and a termination sequence, such as the Nos terminator. These constructs are incorporated into Agrobacterium tumefaciens according to standard methods known in the art, such as electroporation. The transformed A. tumefaciens and are introduced into BY-2 cells. The methods for infecting and selecting the tobacco BY-2 cells are as follows.

Four ml of BY-2 cells which had been cultured for 7 days in 100 mL of modified LS medium (see Imanishi et al., Plant Mol. Biol., 38:1101-1111 (1998)), were subcultured into 100 mL modified LS medium, and cultured for 4 days.

One hundred microliters of A. tumefaciens solution, which had been cultured for 1 day in YEB medium, are added to the 4 mL of cells that had been cultured for 4 days, and the two are cultured together for 40 hours in the dark at 27° C.

After culture, the cells are washed twice with modified LS medium to remove the Agrobacteria. The washed cells are spread on modified LS selection medium containing kanamycin (50 mg/L) and carbenicillin (250 mg/L), and transformed cells are selected.

After culturing for about 2 weeks in the dark at 27° C., the transformed cells are transferred to a fresh modified LS selection medium, and cultured in the dark for 1 week at 27° C.

The transformed cells are then grown in a suspension culture in the dark at 27° C. for 1 week in 30 mL of liquid modified LS medium. 1 mL of the cultured transformed cells are subcultured to 100 mL of modified LS medium. The transformed cells are subcultured every 7 days in the same way as wild-type cells.

Alkaloid Synthesis.

10 mL each of transformed BY-2 cells, which will have been cultured for 7 days and cultured tobacco cells, which will have been transformed using a green fluorescent protein (GFP) expression vector as the control, are washed twice with modified LS medium containing no 2,4-D, and, after addition of modified LS medium containing no 2,4-D to a total of 100 mL, are suspension cultured at 27° C. for 12 hours.

After addition of 100 μL of methyljasmonate (MeJa) which is diluted to 50 μM with DMSO, the cells are suspension cultured for 48 hours at 27° C.

Jasmonate-treated cells are filtered, collected, and freeze dried. Sulfuric acid, 3 mL of 0.1 N, is added to 50 mg of the freeze-dried sample. The mixture is sonicated for 15 minutes, and filtered. A 28% ammonium solution is added to 1 mL of the filtrate, and centrifuged for 10 minutes at 15000 rpm.

One mL of the supernatant is added to an Extrelut-1 column (Merck) and let sit for 5 minutes. This is eluted with 6 mL of chloroform. The eluate is then dried under reduced pressure at 37° C. with an evaporator (Taitec Concentrator TC-8).

The dried sample is dissolved in 50 μL of ethanol solution containing 0.1% dodecane. A gas chromatograph (GC-14B) equipped with a capillary column and an FID detector is used to analyze the samples. A RESTEC Rtx-5Amine column (Restec) is used as the capillary column. The column temperature was maintained at 100° C. for 10 min, elevated to 150° C. at 25° C./min, held at 150° C. for 1 min, elevated to 170° C. at 1° C./min, held at 170° C. for 2 min, elevated to 300° C. at 30° C./min, and then held at 300° C. for 10 min. Injection and detector temperature is 300° C. One μL of each sample is injected, and nicotinic alkaloids are quantified by the internal standard method.

RNA Expression.

To determine whether the reduction of alkaloid accumulation in the transformed BY-2 tobacco cell lines is specifically related to reduction of expression of the nicotine biosynthesis enzyme that is targeted by the dominant negative transcription factor of interest, the levels of expression of nicotine biosynthesis enzymes (e.g., PMT, QPT, ODC, and A622 when dominant negative forms of NtERF32 are employed) are measured in methyl jasmonate treated lines, transgenic lines, and control lines.

Total RNAs are isolated from wild-type and transgenic BY-2 cell lines which are treated with 50 μM MeJA for 48 h. RNA levels of specific genes are determined by RT-PCR methods known in the art.

### Results

It is predicted that transgenic BY-2 cell lines in which one or more nicotine biosynthesis enzymes is suppressed by one or more transcription factors reprogrammed to have dominant-negative functions, jasmonate elicitation will result in reduced accumulation of nicotinic alkaloids, such as nicotine, as compared with non-transformed control cell lines.

Accordingly, these results will show that nicotine biosynthetic pathway transcription factors, when fused to a transcription repressor domain (e.g., an EAR-motif repressor domain, or any other repressor domain as described herein), can act as dominant repressors to produce transgenic cells and plants with a low nicotine phenotype.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All publicly available documents referenced or cited herein, such as patents, patent applications, provisional applications, and publications, including GenBank Accession Numbers, are incorporated by reference in their entirety, including all figures and tables, to the extent they are not inconsistent with the explicit teachings of this specification.

Other embodiments are set forth within the following claims.

What is claimed is:

1. A chimeric nucleic acid construct comprising at least one transcription repressor sequence in translational fusion with at least one transcription factor sequence encoding for a transcription factor or fragment thereof that positively regulates nicotine biosynthesis, wherein the chimeric nucleic acid construct is operably linked to a heterologous nucleic acid.

2. The chimeric nucleic acid of claim 1, wherein the transcription repressor sequence encodes for a transcription repressor selected from the group consisting of: Engrailed (En²⁹⁸) (SEQ ID NO: 11), SRDX (SEQ ID NO: 12), AtERF4 (SEQ ID NO: 13), NtERF3 (SEQ ID NO: 15), SUPERMAN (SEQ ID NO: 17), LEUNIG (SEQ ID NO: 18), and SEUSS (SEQ ID NO: 19).

3. The chimeric nucleic acid of claim 1, wherein the transcription factor sequence encoding for a transcription factor or fragment thereof that positively regulates nicotine biosynthesis is selected from the group consisting of:
(a) the nucleic acid sequence of SEQ ID NO: 1 (NtERF1), SEQ ID NO: 2 (NtERF5), SEQ ID NO: 3 (NtERF10), SEQ ID NO: 4 (NtERF32), SEQ ID NO: 5 (NtERF221), SEQ ID NO: 6 (NtERF241), SEQ ID NO: 7 (NtMYC1a), SEQ ID NO: 8 (NtMYC1b), SEQ ID NO: 9 (NtMYC2a), or SEQ ID NO: 10 (NtMYC2b); and
(b) a nucleic acid sequence that is at least about 80% identical to the nucleic acid sequence of (a), and which encodes for a protein comprising at least one DNA binding domain that positively regulates nicotine biosynthesis.

4. An expression vector comprising the chimeric nucleic acid of claim 1, wherein the operably linked heterologous nucleic acid comprises one or more control sequences suitable for directing expression in a Nicotiana host cell.

5. A Nicotiana host cell transformed with the chimeric nucleic acid construct of claim 1.

6. A genetically engineered nicotinic alkaloid-producing Nicotiana plant comprising a cell comprising the chimeric nucleic acid construct of claim 1.

7. The engineered Nicotiana plant of claim 6, wherein the plant is a Nicotiana tabacum plant.

8. Seeds from the genetically engineered Nicotiana plant of claim 6, wherein the seeds comprise the chimeric nucleic acid construct.

9. A tobacco product comprising the engineered Nicotiana plant of claim 6.

10. A method for reducing nicotine in a Nicotiana plant, comprising:
(a) introducing into a Nicotiana plant an expression vector comprising a chimeric nucleic acid construct comprising, in the 5′ to 3′ direction, a promoter suitable for directing expression in a Nicotiana plant cell operably linked to at least one transcription repressor sequence in translational fusion with at least one transcription factor sequence encoding for a transcription factor or fragment thereof that positively regulates nicotine biosynthesis; and
(b) growing the plant under conditions which allow for the expression of the chimeric nucleic acid;
wherein expression of the chimeric nucleic acid results in production of a dominant negative form of the transcription factor and the plant having a reduced nicotine content as compared to a non-transformed control plant grown under similar conditions.

11. The method of claim 10, wherein the transcription repressor sequence encodes for transcription repressor selected from the group consisting of: Engrailed (En²⁹⁸) (SEQ ID NO: 11), SRDX (SEQ ID NO: 12), AtERF4 (SEQ ID NO: 13), NtERF3 (SEQ ID NO: 15), SUPERMAN (SEQ ID NO: 17), LEUNIG (SEQ ID NO: 18), and SEUSS (SEQ ID NO: 19).

12. The method of claim 10, wherein the transcription factor that positively regulates nicotine biosynthesis is selected from the group consisting of:
(a) the nucleic acid sequence of SEQ ID NO: 1 (NtERF1), SEQ ID NO: 2 (NtERF5), SEQ ID NO: 3 (NtERF10), SEQ ID NO: 4 (NtERF32), SEQ ID NO: 5 (NtERF221), SEQ ID NO: 6 (NtERF241), SEQ ID NO: 7 (NtMYC1a), SEQ ID NO: 8 (NtMYC1b), SEQ ID NO: 9 (NtMYC2a), or SEQ ID NO: 10 (NtMYC2b); and
(b) a nucleic acid sequence that is at least 80% identical to the nucleic acid sequence of (a), and which encodes for a protein comprising at least one DNA binding domain that positively regulates nicotine biosynthesis.

13. A genetically engineered Nicotiana plant produced by the method of claim 10, wherein the plant has reduced expression of nicotine as compared to a control plant.

14. A product comprising the genetically engineered plant of claim 13 or portions thereof, wherein the product has a reduced nicotine content as compared to a product produced from a control plant.

15. The product of claim 14, wherein the product is a reduced-nicotine tobacco product selected from the group consisting of cigarette tobacco, reconstituted tobacco, cigar tobacco, pipe tobacco, cigarettes, cigars, chewing tobacco, snuff, snus, and lozenges.

16. Seeds from the genetically engineered Nicotiana plant of claim 13, wherein the seeds comprise the chimeric nucleic acid construct.

17. A genetically engineered Nicotiana plant having reduced nicotine content as compared to a non-transformed control plant, wherein the plant comprises plant cells comprising:
a chimeric nucleic acid construct comprising, in the 5′ to 3′ direction, a promoter suitable for directing expression in the Nicotiana plant cell operably linked to at least one transcription repressor sequence in translational fusion with at least one transcription factor sequence encoding for a transcription factor or fragment thereof that positively regulates nicotine biosynthesis,
wherein expression of the chimeric nucleic acid results in production of a dominant negative form of the transcription factor and the plant having a reduced nicotine content as compared to a non-transformed control plant grown under similar conditions.

18. The plant of claim 17, wherein the transcription repressor sequence encodes for a transcription repressor selected from the group consisting of: Engrailed (En²⁹⁸) (SEQ ID NO: 11), SRDX (SEQ ID NO: 12), AtERF4 (SEQ ID NO: 13), NtERF3 (SEQ ID NO: 15), SUPERMAN (SEQ ID NO: 17), LEUNIG (SEQ ID NO: 18), and SEUSS (SEQ ID NO: 19).

19. The plant of claim 17, wherein the transcription factor that positively regulates nicotine biosynthesis is selected from the group consisting of:
(a) the nucleic acid sequence of SEQ ID NO: 1 (NtERF1), SEQ ID NO: 2 (NtERF5), SEQ ID NO: 3 (NtERF10), SEQ ID NO: 4 (NtERF32), SEQ ID NO: 5 (NtERF221), SEQ ID NO: 6 (NtERF241), SEQ ID NO: 7 (NtMYC1a), SEQ ID NO: 8 (NtMYC1b), SEQ ID NO: 9 (NtMYC2a), or SEQ ID NO: 10 (NtMYC2b); and
(b) a nucleic acid sequence that is at least about 80% identical to the nucleic acid sequence of (a), and which encodes for a protein comprising at least one DNA binding domain that positively regulates nicotine biosynthesis.

20. A product comprising the genetically engineered plant of claim 17 or portions thereof, wherein the product has a reduced nicotine content as compared to a product produced from a control plant.

21. The product of claim 20, wherein the product is a reduced-nicotine tobacco product selected from the group consisting of cigarette tobacco, reconstituted tobacco, cigar tobacco, pipe tobacco, cigarettes, cigars, chewing tobacco, snuff, snus, and lozenges.

22. Seeds from the genetically engineered Nicotiana plant of claim 17, wherein the seeds comprise the chimeric nucleic acid construct.

23. A method of making a genetically engineered Nicotiana plant cell having reduced nicotine content, the method comprising:
introducing into a Nicotiana plant cell an expression vector comprising a chimeric nucleic acid construct comprising, in the 5′ to 3′ direction, a promoter suitable for directing expression in a Nicotiana plant cell operably linked to at least one transcription repressor sequence in translational fusion with at least one transcription factor sequence encoding for a transcription factor or fragment thereof that positively regulates nicotine biosynthesis;
wherein expression of the chimeric nucleic acid results in production of a dominant negative form of the transcription factor and the plant cell having a reduced nicotine content as compared to a non-transformed control plant cell.

24. The method of claim 23, wherein the transcription repressor sequence encodes for transcription repressor selected from the group consisting of: Engrailed (En²⁹⁸) (SEQ ID NO: 11), SRDX (SEQ ID NO: 12), AtERF4 (SEQ ID NO: 13), NtERF3 (SEQ ID NO: 15), SUPERMAN (SEQ ID NO: 17), LEUNIG (SEQ ID NO: 18), and SEUSS (SEQ ID NO: 19).

25. The method of claim 23, wherein the transcription factor that positively regulates nicotine biosynthesis is selected from the group comprising of:
(a) the nucleic acid sequence of SEQ ID NO: 1 (NtERF1), SEQ ID NO: 2 (NtERF5), SEQ ID NO: 3 (NtERF10), SEQ ID NO: 4 (NtERF32), SEQ ID NO: 5 (NtERF221), SEQ ID NO: 6 (NtERF241), SEQ ID NO: 7 (NtMYC1a), SEQ ID NO: 8 (NtMYC1b), SEQ ID NO: 9 (NtMYC2a), or SEQ ID NO: 10 (NtMYC2b); and
(b) a nucleic acid sequence that is at least about 80% identical to the nucleic acid sequence of (a), and which encodes for a protein comprising at least one DNA binding domain that positively regulates nicotine biosynthesis.

26. The method of claim 23, wherein the Nicotiana plant cell is Nicotiana tabacum.
SEQUENCE LISTING
SEQ ID NO: 1 (711 bp)
NtERF1 ORF (GenBank Accession No.: D38123.1)

| | |
|---|---|
| 1 | atgaatcaac caatttatac agagttgccg ccggcgaatt ttccgggaga atttccggtg |
| 61 | taccgccgga attcaagctt cagtcgtcta atcccatgtt taactgaaac atggggcgac |
| 121 | ttaccactaa aagtcgacga ttctgaagat atggtaattt atactctctt aaaagacgct |
| 181 | cttaacgtcg gatggtcgcc gtttaatttc agcgccggcg aagtaaaatc ggagcagagg |
| 241 | gaggaagaaa ttgtggtttc tccggcggag acgacggccg cgccggcggc tgagttacct |
| 301 | aggggaaggc attacagagg tgttagacga cggccgtggg ggaaatttgc ggcggagatt |
| 361 | agggatccgg cgaagaatgg agctagggtt tggcttggaa catacgaaac agatgaagaa |
| 421 | gctgcaattg cttatgataa agcggcttat agaatgcgcg gttcaaaggc tcatttaaat |
| 481 | tttccacata gaatcggttt aaatgaaccg gaaccggttc gagttacggc gaaaagacga |
| 541 | gcatcgcctg aaccggctag ttcgtcggaa aatagttcac ctaaacggag aagaaaggct |
| 601 | gttgcaactg agaaatctga agcagtagaa gtggagagta aatcaaatgt tttgcaaact |
| 661 | ggatgtcaag ttgaactatt gacacgtcga catcaattat tagtcagtta a |

SEQ ID NO: 2 (705 bp)
NtERF5/NtERF121 ORF (GenBank Accession No.: AY655738.1)

| | |
|---|---|
| 1 | atgtcaagta actcaagccc actagaaata gacacttcat tttcacattc caacttcttc |
| 61 | tttctccaag atcaatcacc aattttacaa tgggatgatg atcttttctt caatgatcca |
| 121 | tggtttgatg atgatcaatc accaattata ccatgtaact cagagaaaga tgaaaatcat |
| 181 | caagtatttg aagaatcctc agacaataca atcatgtcaa aaggaagtag ccatggtcaa |
| 241 | gaattagaag aggtaacatc ccaagaagaa aaagaaaaag aagaagaaga aaaacactat |
| 301 | ataggagtta gaaaaaggcc atggggtaaa tatgcagcag aaataagaga ttcaacaaga |
| 361 | aatggaatta gggtttggtt agggacattt gatacagctg aagaagctgc tttagcttat |
| 421 | gatcaagctg cattatcgat gagaggtcct tggtctcttc ttaattttcc attggagaaa |
| 481 | gtcaagaaat cacttgaaaa aattgagtat tcttgtaaag atggattgtc tcctgctgct |
| 541 | gttctaaaag ctactcataa aacaaggaga gtgaagcata aaagaagtag tagaaagaaa |
| 601 | aagaataaag aaactcataa tgttattgtt tttgaggact tgggtgctga gttattagaa |
| 661 | gagcttttaa tgacttcatc acaacattcg tgtcgaaggg actga |

SEQ ID NO: 3 (858 bp)
NtERF10/JAP1 gene (GenBank Accession No.: CQ808845.1)

| | |
|---|---|
| 1 | acgggggggg gggggggggg ggacttgaag actgggaagc tccattaacg agctccgaca |
| 61 | actcaacagc ctctgattta agccgaagca atagcattga gtccaacatg tttcctaatt |
| 121 | gcttgcccaa tgaatataat tatacagctg atatgttttt taacgatatc tttaatgaag |
| 181 | gcattgttgg ctatggattt gagccagctt ctgaatttac actccccagt atcaaattgg |
| 241 | agccagaaat gactgtacaa tcacctgcaa tatggaattt accggagttt gtggcgccgc |
| 301 | cggagacggc ggcggaggtg aaactggaac caccggcgcc gcaaaaggca aagcattata |
| 361 | ggggagtgag agtgaggccg tgggggaagt ttgcagcgga aattagggat ccggcaaaga |
| 421 | atggggcaag ggtgtggctg ggtacgtatg agacggcaga ggacgcagcg tttgcttatg |
| 481 | acaaggcggc gtttcgcatg cgggggtcac gtgcattgct taatttcccg ttaaggatta |
| 541 | attctggtga gcctgatccc attagagttg gttctaaaag gtcatcaatg tcgccggagt |
| 601 | attcttcttc ttcatcgtcg tcggcgtcgt cgccgaagag gaggaagaag gtatctcaag |
| 661 | ggacggagct aacggtgtta taggtcccaa ctgggttctg tgtagtgatt aagaaaaata |
| 721 | gaattagtcg agggaatttg ttttttactt ggctgaagta atgaatttgt tatttattta |
| 781 | ttttttgact gtggttgaaa ttgaatcaaa aaaaaaaaaa aaaaagtact agtcgacgcg |
| 841 | tggcctagta gtagtaga |

SEQ ID NO: 4 (702 bp)
NtERF32/EREBP2/ERF2 ORF (GenBank Accession No.: D38126.1)

| | |
|---|---|
| 1 | atgtatcaac caatttcgac cgagctacct ccgacgagtt tcagtagtct catgccatgt |
| 61 | ttgacggata catggggtga cttgccgtta aaagttgatg attccgaaga tatggtaatt |
| 121 | tatgggctct taagtgacgc tttaactgcc ggatggacgc cgtttaattt aacgtccacc |
| 181 | gaaataaaag ccgagccgag ggaggagatt gagccagcta cgattcctgt tccttcagtg |
| 241 | gctccacctg cggagactac gacggctcaa gccgttgttc ccaaggggag gcattatagg |
| 301 | ggcgttaggc aaaggccgtg ggggaaattt gcggcggaaa taagggaccc agctaaaaac |
| 361 | ggcgcacggg tttggctagg gacttatgag acggctgaag aagccgcgct cgcttatgat |
| 421 | aaagcagctt acaggatgcg cggctccaag gctctattga attttccgca taggatcggc |
| 481 | ttaaatgagc ctgaaccggt tagactaacc gctaagagac gatcacctga accggctagc |
| 541 | tcgtcaatat catcggcttt ggaaaatggc tcgccgaaac ggaggagaaa agctgtagcg |
| 601 | gctaagaagg ctgaattaga agtgcaaagc cgatcaaatg ctatgcaagt tgggtgccag |
| 661 | atggaacaat ttccagttgg cgagcagcta ttagtcagtt aa |

SEQ ID NO: 5 (924 bp)
NtERF221/ORC1 gene (GenBank Accession No.: CQ808982.1)

| | |
|---|---|
| 1 | atccagaatt aataaaccct agtaagtgaa agtgaaagaa actactcatc caaatatcta |
| 61 | tagaaaagta aatgaatccc gctaatgcaa ccttctcttt ctctgagctt gatttccttc |
| 121 | aatcaataga aaaccatctt ctgaattatg attccgattt ttctgaaatt ttttcgccga |
| 181 | tgagttcaag taacgcattg cctaatagtc ctagctcaag ttttggcagc ttcccttcag |
| 241 | cagaaaatag cttggatacc tctctttggg atgaaaactt tgaggaaaca atacaaaatc |
| 301 | tcgaagaaaa gtccgagtcc gaggaggaaa caaaggggca tgtcgtggcg cgtgagaaaa |
| 361 | acgcgacaca agattggaga cggtacatag gagttaaacg gcggccgtgg gggacgtttt |
| 421 | cggcggagat aagggacccg gagagaagag gcgcgagatt atggctagga acttacgaga |
| 481 | ccccagagga cgcagcattg gcttacgatc aagccgcttt caaaatccgc ggctcgagag |
| 541 | ctcggctcaa ttttcctcac ttaattggat caaacattcc taagccggct agagttacag |
| 601 | cgagacgtag ccgtacgcgc tcaccccagc catcgtcttc ttcatgtacc tcatcatcag |
| 661 | aaaatgggac aagaaaaagg aaaatagatt tgataaattc catagccaaa gcaaaattta |
| 721 | ttcgtcatag ctggaaccta caaatgttgc tataactgta tttaatttgg aaggaattaa |
| 781 | ttaaggttat tctatgtctt tgtattagaa tttagaataa ttccctaaag ctcctgaaga |
| 841 | acgaaacttg taaacatctc tctgtctccg tatcatgttc taatttaaca tgaaattaca |
| 901 | tgagcgcaaa aaaaaaaaaa aaaa |

SEQ ID NO: 6 (741 bp)
NtERF241 ORF
ATGTATCAACCCATTTCTACAGAATTCCCAGTATATCACCGGACTTCAAGTTTCAGTAGTCTCAT
GCCATGTTTGACGGATACTTGGGGTGACTTGCCGTTAAAAGTTGATGATTCCGAAGATATGGTAA
TTTATGGGCTCTTAAGTGACGCTTTAACTACCGGATGGACGCCGTTTAATTTAACGTCCACCGAA
ATAAAAGCCGAGCCGAGGGAAGAGATTGAGCCAGCTACGAGTCCTGTTCCTTCAGTGGCTCCACC
CGCGGAGACTACGACGGCTCAAGCCGTCGTGCCCAAGGGAAGGCATTATAGGGGCGTCAGGCAAA
GGCCGTGGGGGAAATTTGCGGCGGAAATAAGGGACCCAGCTAAAAATGGCGCACGGGTTTGGCTA
GGGACTTATGAGACGGCTGAAGAAGCCGCGCTCGCTTATGATAAAGCAGCTTACAGGATGCGCGG
CTCCAAGGCTCTATTGAATTTTCCGCATAGGATCGGCTTAAATGAGCCTGAACCGGTTAGGCTGA
CCGTTAAGAGACGATCACCTGAACCGGCCGTTAAGAGACGATCACCTGAACCGGCTAGCTCGTCA
ATATCACCGGCTTCGGAAAATAGCTTGCCGAAGCGGAGGAGAAAAGCTGTAGCGGCTAAGCAAGC
TGAATTAGAAGTGCAGAGCCGATCAAATGTAATGCAAGTTGGGTGCCAAATGGAACAATTTCCAG
TTGGCGAGCAGCTATTAGTTAGTTAA
SEQ ID NO: 7 (2046 bp)
NtMYC1a ORF (GenBank Accession No.: GQ859158.1)

| | |
|---|---|
| 1 | atgactgatt acagcttacc caccatgaat ttgtggaata ctagtggtac taccgatgac |
| 61 | aacgttacta tgatggaagc ttttatgtct tctgatctca cttcattttg ggctacttct |
| 121 | aattctactg ctgttgctgc tgttacctct aattctaatc atattccagt taatacccca |
| 181 | acggttcttc ttccgtcttc ttgtgcctct actgtcacag ctgtggctgt cgatgcttca |
| 241 | aaatccatgt cttttttcaa ccaagaaacc cttcaacagc gtcttcaaac gctcattgat |
| 301 | ggtgctcgtg agacgtggac ctatgccatc ttttggcagt catccgccgt tgatttaacg |
| 361 | agtccgtttg tgttgggctg gggagatggt tactacaaag gtgaagaaga taaagccaat |
| 421 | aggaaattag ctgtttcttc tcctgcttat atagctgagc aagaacaccg gaaaaaggtt |
| 481 | ctccgggagc tgaattcgtt gatttccggc acgcaaaccg gcactgatga tgccgtcgat |
| 541 | gaagaagtta ccgacactga atggttcttc cttatttcca tgacccagtc gtttgttaac |
| 601 | ggaagtgggc ttccgggtca ggccttatac aattccagcc ctatttgggt cgccggagca |
| 661 | gagaaattgg cagcttccca ctgcgaacgg gctcggcagg cccagggatt cgggcttcag |
| 721 | acgatggttt gtattccttc agcaaacggc gtggttgaat tgggctccac ggagttgatt |
| 781 | attcagagtt ctgatctcat gaacaaggtt agagtattgt ttaacttcaa taatgatttg |
| 841 | ggctctggtt cgtgggctgt gcaacccgag agcgatccgt ccgctctttg gctcactgat |
| 901 | ccatcgtctg cagctgtaca agtcaaagat ttaaatacag ttgaggcaaa ttcagttcca |
| 961 | tcaagtaata gtagtaagca agttgtattt gataatgaga ataatggtca cagttgtgat |
| 1021 | aatcagcaac agcaccattc tcggcaacaa acacaaggat tttttacaag ggagttgaac |
| 1081 | ttttcagaat tcgggtttga tggaagtagt aataatagga atgggaattc atcactttct |
| 1141 | tgcaagccag agtcggggga aatcttgaat tttggtgata gcactaagaa aagtgcaaat |
| 1201 | gggaacttat tttccggtca gtcccatttt ggtgcagggg aggagaataa gaagaagaaa |
| 1261 | aggtcacctg cttccagagg aagcaatgaa gaaggaatgc tttcatttgt ttcaggtaca |
| 1321 | atcttgcctg cagcttctgg tgcgatgaag tcaagtggat gtgtcggtga agactcctct |
| 1381 | gatcattcgg atcttgaggc ctcagtggtg aaagaagctg aaagtagtag agttgtagaa |
| 1441 | cccgaaaaga ggccaaagaa gcgaggaagg aagccagcaa atggacgtga ggaacctttg |
| 1501 | aatcacgtcg aagcagagag gcaaaggaga gagaaattaa accaaaggtt ctacgcttta |
| 1561 | agagctgttg ttccgaatgt gtccaagatg gacaaggcat cactgcttgg agatgcaatt |
| 1621 | tcatatatta atgagctgaa gttgaagctt caaactacag aaacagatag agaagacttg |
| 1681 | aagagccaaa tagaagattt gaagaaagaa ttagatagta aagactcaag gcgccctggt |
| 1741 | cctccaccac caaatcaaga tcacaagatg tctagccata ctggaagcaa gattgtagat |
| 1801 | gtggatatag atgttaagat aattggatgg gatgcgatga ttcgtataca atgtaataaa |
| 1861 | aagaaccatc cagctgcaag gttaatggta gccctcaagg agttagatct agatgtgcac |
| 1921 | catgccagtg tttcagtggt gaatgatttg atgatccaac aagccacagt gaaaatgggt |
| 1981 | agcagacttt acacggaaga gcaacttagg atagcattga catccagagt tgctgaaaca |
| 2041 | cgctaa |

SEQ ID NO: 8 (2040 bp)
NtMYC1b ORF (GenBank Accession No.: GQ859159.1)

| | |
|---|---|
| 1 | cgcagacccc tcttttcacc catttctctc tctctctctc tctctctctc tatatatata |
| 61 | tatatctttc acgccaccat atccaactgt ttgtgctggg tttatggaat gactgattac |
| 121 | agcttaccca ccatgaattt gtggaatact agtggtacta ccgatgacaa cgtttctatg |
| 181 | atggaatctt ttatgtcttc tgatctcact tcattttggg ctacttctaa ttctactact |
| 241 | gctgctgtta cctctaattc taatcttatt ccagttaata ccctaactgt tcttcttccg |
| 301 | tcttcttgtg cttctactgt cacagctgtg gctgtcgatg cttcaaaatc catgtctttt |
| 361 | ttcaaccaag aaactcttca gcagcgtctt caaaccctca ttgatggtgc tcgtgagacg |
| 421 | tggacctatg ccatcttttg gcagtcatcc gtcgttgatt tatcgagtcc gtttgtgttg |
| 481 | ggctggggag atggttacta caaaggtgaa gaagataaag ccaataggaa attagctgtt |
| 541 | tcttctcctg cttatattgc tgagcaagaa caccgaaaaa aggttctccg ggagctgaat |
| 601 | tcgttgatct ccggcacgca aaccggcact gatgatgccg tcgatgaaga agttaccgac |
| 661 | actgaatggt tcttccttat ttccatgacc caatcgtttg ttaacggaag tgggcttccg |
| 721 | ggtcaggcct tatacaattc cagccctatt tgggtcgccg gagcagagaa attggcagct |
| 781 | tcccactgcg aacgggctcg gcaggcccag ggattcgggc ttcagacgat ggtttgtatt |
| 841 | ccttcagcaa acggcgtggt tgaattgggc tccacggagt tgataatcca gagttgtgat |
| 901 | ctcatgaaca aggttagagt attgtttaac ttcaataatg atttgggctc tggttcgtgg |
| 961 | gctgtgcagc ccgagagcga tccgtccgct ctttggctca ctgatccatc gtctgcagct |
| 1021 | gtagaagtcc aagatttaaa tacagttaag gcaaattcag ttccatcaag taatagtagt |
| 1081 | aagcaagttg tgtttgataa tgagaataat ggtcacagtt ctgataatca gcaacagcag |
| 1141 | cattctaagc atgaaacaca aggatttttc acaagggagt tgaatttttc agaatttggg |
| 1201 | tttgatggaa gtagtaataa taggaatggg aattcatcac tttcttgcaa gccagagtcg |
| 1261 | ggggaaatct tgaattttgg tgatagtact aagaaaagtg caaatgggaa cttattttcg |
| 1321 | ggtcagtccc attttggggc aggggaggag aataagaaca agaaaaggtc acctgcttcc |
| 1381 | agaggaagca atgaagaagg aatgctttca tttgtttcgg gtacaatctt gcctgcagct |
| 1441 | tctggtgcga tgaagtcaag tggaggtgta ggtgaagact ctgatcattc ggatcttgag |
| 1501 | gcctcagtgg tgaaagaagc tgaaagtagt agagttgtag aacccgaaaa gaggccaaag |
| 1561 | aagcgaggaa ggaagccagc aaatggacgg gaggaacctt tgaatcacgt cgaagcagag |
| 1621 | aggcaaagga gagagaaatt aaaccaaagg ttctacgcat taagagctgt tgttccgaat |
| 1681 | gtgtccaaga tggacaaggc atcactgctt ggagatgcaa tttcatatat taatgagctg |
| 1741 | aagttgaagc ttcaaaatac agaaacagat agagaagaat tgaagagcca aatagaagat |
| 1801 | ttaaagaaag aattagttag taaagactca aggcgccctg gtcctccacc atcaaatcat |
| 1861 | gatcacaaga tgtctagcca tactggaagc aagattgtag acgtggatat agatgttaag |
| 1921 | ataattggat gggatgcgat gattcgtata caatgtaata aaaagaatca tccagctgca |
| 1981 | aggttaatgg tagccctcaa ggagttagat ctagatgtgc accatgccag tgtttcagtg |
| 2041 | gtgaacgatt tgatgatcca acaagccact gtgaaaatgg gtagcagact ttacacggaa |
| 2101 | gagcaactta ggatagcatt gacatccaga gttgctgaaa cacgctaa |

SEQ ID NO: 9 (1980 bp)
NtMYC2a ORF (GenBank Accession No.: GQ859160.1)

| | |
|---|---|
| 1 | atgacggatt atagaatacc aacgatgact aatatatgga gcaatactac atccgatgat |
| 61 | aatatgatgg aagctttttt atcttctgat ccgtcgtcgt tttggcccgg aacaactact |
| 121 | acaccaactc cccggagttc agtttctcca gcgccggcgc cggtgacggg gattgccgga |
| 181 | gacccattaa agtctatgcc atatttcaac caagagtcac tgcaacagcg actccagact |
| 241 | ttaatcgatg gggctcgcaa agggtggacg tatgccatat tttggcaatc gtctgttgtg |
| 301 | gatttcgcga gcccctcggt tttggggtgg ggagatgggt attataaagg tgaagaagat |
| 361 | aaaaataagc gtaaaacggc gtcgttttcg cctgacttta tcacggaaca agcacaccgg |
| 421 | aaaaaggttc tccgggagct gaattcttta atttccggca cacaaaccgg tggtgaaaat |
| 481 | gatgctgtag atgaagaagt aactgatact gaatggtttt ttctgatttc catgacacaa |
| 541 | tcgtttgtta acggaagcgg gcttccgggc ctggcgatgt atagttcaag cccgatttgg |
| 601 | gttactggaa cagagagatt agctgtttct cactgtgaac gggcccgaca ggcccaaggt |
| 661 | ttcgggcttc agactattgt ttgtattcct tcagctaatg gtgttgttga gctcgggtca |
| 721 | actgagttga tattccagac tgctgattta atgaacaagg ttaaagtttt gtttaatttt |
| 781 | aatattgata tgggtgcgac tacgggctca ggatcgggct catgtgctat tcaggccgag |
| 841 | cccgatcctt cagccctttg gctgactgat ccggcttctt cagttgtgga agtcaaggat |
| 901 | tcgtcgaata cagttccttc aaggaatacc agtaagcaac ttgtgtttgg aaatgagaat |
| 961 | tctgaaaatg ttaatcaaaa ttctcagcaa acacaaggat ttttcactag ggagttgaat |
| 1021 | ttttccgaat atggatttga tggaagtaat actcggtatg gaaatgggaa tgcgaattct |
| 1081 | tcgcgttctt gcaagcctga gtctggtgaa atcttgaatt ttggtgatag tactaagagg |
| 1141 | agtgcttgca gtgcaaatgg gagcttgttt tcgggccaat cacagttcgg gcccgggcct |
| 1201 | gcggaggaga acaagaacaa gaacaagaaa aggtcacctg catcaagagg aagcaacgat |
| 1261 | gaaggaatcc tttcatttgt ttcgggtgtg attttgccaa gttcaaacac ggggaagtcc |
| 1321 | ggtggaggtg gcgattcgga tcaatcagat ctcgaggctt cggtggtgaa ggaggcggat |
| 1381 | agtagtagag ttgtagaccc cgagaagaag ccgaggaaac gagggaggaa accggctaac |
| 1441 | gggagagagg agccattgaa tcatgtggag gcagagagac aaaggaggga gaaattgaat |
| 1501 | caaagattct atgcacttag agctgttgta ccaaatgtgt caaaaatgga taaagcatca |
| 1561 | cttcttggtg atgcaattgc atttatcaat gagttgaaat caaaggttca gaattctgac |
| 1621 | tcagataaag aggacttgag gaaccaaatc gaatctttaa ggaatgaatt agccaacaag |
| 1681 | ggatcaaact ataccggtcc tcccccgtca aatcaagaac tcaagattgt agatatggac |
| 1741 | atcgacgtta aggtgatcgg atgggatgct atgattcgta tacaatctaa taaaaagaac |
| 1801 | catccagccg cgaggttaat gaccgctctc atggaattgg acttagatgt gcaccatgct |
| 1861 | agtgtttcag ttgtcaacga gttgatgatc caacaagcga ctgtgaaaat gggaagccgg |
| 1921 | ctttacacgc aagaacaact tcggatatca ttgacatcca gaattgctga atcgcgatga |

SEQ ID NO: 10 (1977 bp)
NtMYC2b ORF (GenBank Accession No.: GQ859161)

| | |
|---|---|
| 1 | atgacggact atagaatacc aacgatgact aatatatgga gcaatacaac atccgacgat |
| 61 | aacatgatgg aagctttttt atcttctgat ccgtcgtcgt tttgggccgg aacaaataca |
| 121 | ccaactccac ggagttcagt ttctccggcg ccggcgccgg tgacggggat tgccggagac |
| 181 | ccattaaagt cgatgccgta tttcaaccaa gagtcgctgc aacagcgact ccagacgtta |
| 241 | atcgacgggg ctcgcgaagc gtggacttac gccatattct ggcaatcgtc tgttgtggat |
| 301 | ttcgtgagcc cctcggtgtt ggggtgggga gatggatatt ataaaggaga agaagacaag |
| 361 | aataagcgta aaacggcggc gttttcgcct gattttatta cggagcaaga acaccggaaa |
| 421 | aaagttctcc gggagctgaa ttctttaatt tccggcacac aaactggtgg tgaaaatgat |
| 481 | gctgtagatg aagaagtaac ggatactgaa tggttttttc tgatttcaat gactcaatcg |
| 541 | tttgttaacg gaagcgggct tccgggcctg gctatgtaca gctcaagccc gatttgggtt |
| 601 | actggaagag aaagattagc tgcttctcac tgtgaacggg cccgacaggc ccaaggtttc |
| 661 | gggcttcaga ctatggtttg tattccttca gctaatggtg ttgttgagct cgggtcaact |
| 721 | gagttgatat tccagagcgc tgatttaatg aacaaggtta aaatcttgtt tgattttaat |
| 781 | attgatatgg gcgcgactac gggctcaggt tcgggctcat gtgctattca ggctgagccc |
| 841 | gatccttcaa ccctttggct tacggatcca ccttcctcag ttgtggaagt caaggattcg |
| 901 | tcgaatacag ttccttcaag taatagtagt aagcaacttg tgtttggaaa tgagaattct |
| 961 | gaaaatgtta atcaaaattc tcagcaaaca caaggatttt tcactaggga gttgaatttt |
| 1021 | tccgaatatg gatttgatgg aagtaatact aggagtggaa atgggaatgt gaattcttcg |
| 1081 | cgttcttgca agcctgagtc tggcgaaatc ttgaattttg gtgatagtac taagagaaat |
| 1141 | gcttcaagtg caaatgggag cttgttttcg ggccaatcgc agttcggtcc cgggcctgcg |
| 1201 | gaggagaaca agaacaagaa caagaaaagg tcacctgcat caagaggaag caatgaagaa |
| 1261 | ggaatgcttt catttgtttc gggtgtgatc ttgccaagtt caaacacggg gaagtccggt |
| 1321 | ggaggtggcg attcggatca ttcagatctc gaggcttcgg tggtgaagga ggcggatagt |
| 1381 | agtagagttg tagaccccga gaagaggccg aggaaacgag gaaggaaacc ggctaacggg |
| 1441 | agagaggagc cattgaatca tgtggaggca gagaggcaaa ggagggagaa attgaatcaa |
| 1501 | agattctatg cacttagagc tgttgtacca aatgtgtcaa aaatggataa agcatcactt |
| 1561 | cttggtgatg caattgcatt tatcaatgag ttgaaatcaa aggttcagaa ttctgactca |
| 1621 | gataaagatg agttgaggaa ccaaattgaa tctttaagga atgaattagc caacaaggga |
| 1681 | tcaaactata ccggtcctcc accgccaaat caagatctca agattgtaga tatggatatc |
| 1741 | gacgttaaag tcatcggatg ggatgctatg attcgtatac aatctaataa aaagaaccat |
| 1801 | ccagccgcga ggttaatggc cgctctcatg gaattggact tagatgtgca ccatgctagt |
| 1861 | gtttcagttg tcaacgagtt gatgatccaa caagcgacag tgaaaatggg gagccggctt |
| 1921 | tacacgcaag agcagcttcg gatatcattg acatccagaa ttgctgaatc gcgatga |

```
SEQ ID NO: 11 (298 AA)
Engrailed (En²⁹⁸) protein from Drosophila (the first 298 amino acids encoded by the
Engrailed gene from Drosophila)
Met Met Met Asn Ala Phe Ile Glu Pro Ala Gln His His Leu Ala Ser
1 5 10 15
Tyr Gly Leu Arg Met Ser Pro Asn Thr Thr Ala Ser Asn Ser Asn Ala
                        20 25 30
Gln Gln Gln Gln Gln Gln Gln Leu Glu Met Thr Gln Gln Gln Gln Gln
                35 40 45
Gln Gln Gln Gln Gln Gln Gln Gln Gln Gln Gln Asp Gln Glu Ser Ala
        50 55 60
Ala Ala Thr Ala Ala Ala Tyr Gln Asn Ser Gly Tyr Gly His Phe Asn
65 70 75 80
Ser Tyr Ala Ser Arg Asp Phe Leu Leu Gly Arg Arg Glu Ala Glu Tyr
                                85 90 95
Gly Val Ala Gly Ser Ala Gly Gln Ala Ser Ala Ala Ala Asp Ser Met
                        100 105 110
Leu Phe Ser Gly Phe Pro Ala Gln Ala Ala Glu Leu Gly Ser Gly Phe
                115 120 125
Gly Gln His Pro Phe His Ser His His His His His Gln Met Arg Met
        130 135 140
Gly Met Ala Asp Ala Tyr Ala Ala Gly His Pro Tyr Asn His His Gly
145 150 155 160
Asn Phe Pro Thr Ala Ala Val His His Pro Val Val His His Pro Ser
                                165 170 175
His His Ala Met Ser Ala Met His Pro Ala Gly Ala Gly Ala Phe Leu
                        180 185 190
Arg Tyr Met Arg His Gln Pro Ala Ser Ser Ala Ser Ser Val Lys Gln
                195 200 205
Glu Met Gln Cys Leu Trp Ile Asp Pro Asp Gln Pro Gly Leu Val Pro
        210 215 220
Pro Gly Gly Arg Lys Thr Cys Asn Lys Val Phe His Ser Met His Glu
225 230 235 240
Ile Val Thr His Leu Thr Val Glu His Val Gly Gly Pro Glu Cys Thr
                                245 250 255
Thr His Ala Cys Phe Trp Val Gly Cys Ser Arg Asn Gly Arg Pro Phe
                        260 265 270
Lys Ala Lys Tyr Lys Leu Val Asn His Ile Arg Val His Thr Gly Glu
                275 280 285
Lys Pro Phe Ala Cys Pro His Pro Gly Cys
        290 295
SEQ ID NO: 12 (12 AA)
EAR repressor domain protein also known as SRDX (A 12-amino acid sequence of the
Arabidopsis Ethylene-responsive transcription factor 4 (ERF4))
LDLDLELRLGFA
SEQ ID NO: 13 (24 AA)
EAR repressor domain protein also known as AtERF4 (A 24-amino acid sequence of the
Arabidopsis Ethylene-responsive transcription factor 4 (ERF4))
EGGMEKRSQLLDLDLNLPPPSEQA
SEQ ID NO: 14 (225 AA)
Nicotiana tabacum ethylene responsive element binding factor 3 (NtERF3) protein
```

| | |
|---|---|
| 1 | mavknkvsng nlkggnvktd gvkevhyrgv rkrpwgryaa eirdpgkksr vwlgtfdtae |
| 61 | eaakaydtaa refrgpkakt nfpsptenqs pshsstvess sgengvhapp hapleldltr |
| 121 | rlgsvaadgg dncrrsgevg ypifhqqptv avlpngqpvl lfdslwragv vnrpqpyhvt |
| 181 | pmgfngvnag vgptvsdsss aveenqydgk rgidldlnla ppmef |

SEQ ID NO: 15 (35 AA)
EAR repressor domain of NtERF3 protein (amino acids 191-225)
VGPTVSDSSSAVEENQYDGKRGIDLDLNLAPPMEF
SEQ ID NO: 16 (204 AA)
Arabidopsis thaliana SUPERMAN (SUP) protein

| | |
|---|---|
| 1 | mersnsielr nsfygrarts pwsygdydnc qqdhdyllgf swpprsytcs fckrefrsaq |
| 61 | algghmnvhr rdrarlrlqq spsssstpsp pypnpnysys tmansppphh spltlfptls |
| 121 | ppsspryrag lirslspksk htpenacktk kssllveage atrftskdac kilrndeiis |
| 181 | leleigline seqdldlelr lgfa |

SEQ ID NO: 17 (30 AA)
EAR repressor domain of SUPERMAN (SUP) protein (amino acids 175-204)
NDEIISLELEIGLINESEQDLDLELRLGFA
SEQ ID NO: 18 (969 AA)
LEUNIG transcriptional co-repressor protein (Arabidopsis thaliana)

| | |
|---|---|
| 1 | msqtnweadk mldvyihdyl vkrdlkataq afqaegkvss dpvaidapgg flfewwsvfw |
| 61 | difiartnek hsevaasyie tqmikareqq lqqsqhpqvs qqqqqqqqqq iqmqqlllqr |
| 121 | aqqqqqqqqq qhhhhqqqqq qqqqqqqqqq qqqqqhqnqp psqqqqqqst pqhqqqptpq |
| 181 | qqpqrrdgsh langsanglv gnnsepvmrq npgsgsslas kayeervkmp tqresldeaa |
| 241 | mkrfgdnvgq lldpshasil ksaaasgqpa gqvlhstsgg mspqvqtrnq qlpgsavdik |
| 301 | seinpvltpr tavpegslig ipgrfavlsv sfqlvkrvkc sistsahknl lrindwfpvs |
| 361 | gsnqgsnnlt lkgwpltgfd qlrsgllqqq kpfmqsqsfh qlnmltpqhq qqlmlaqqnl |
| 421 | nsqsvseenr rlkmllnnrs mtlgkdglgs svgdvlpnvg sslqpggsll prgdtdmllk |
| 481 | lkmallqqqq qnqqqgggnp pqpqpqpqpl nqlaltnpqp qssnhsihqq eklggggsit |
| 541 | mdgsisnsfr gneqvlknqs grkrkqpvss sgpanssgta ntagpspssa pstpsthtpg |
| 601 | dvismpnlph sggssksmmm fgtegtgtlt spsnqladmd rfvedgsldd nvesflsqed |
| 661 | gdqrdavtrc mdvskgftft evnsvrastt kvtcchfssd gkmlasaghd kkavlwytdt |
| 721 | mkpkttleeh tamitdirfs psqlrlatss fdktvrvwda dnkgyslrtf mghssmvtsl |
| 781 | dfhpikddli cscdndneir ywsinngsct rvykggstqi rfqprvgkyl aassanlvnv |
| 841 | ldvetqairh slqghanpin svcwdpsgdf lasvsedmvk vwtlgtgseg ecvhelscng |
| 901 | nkfqscvfhp aypsllvigc yqslelwnms enktmtlpah eglitslavs tatglvasas |
| 961 | hdklvklwk |

SEQ ID NO: 19 (877 AA)
SEUSS transcriptional co-repressor protein (Arabidopsis thaliana)

| | |
|---|---|
| 1 | mvpseppnpv gggenvppsi lggqggaplp sqpafpslvs prtqfgnnms msmlgnapni |
| 61 | ssllnnqsfv ngipgsmism dtsgaesdpm snvgfsglss fnassmvspr ssgqvqgqqf |
| 121 | snvsanqlla eqqrnkkmet qsfqhgqqqs mqqqfstvrg gglagvgpvk mepgqvsndq |
| 181 | qhgqvqqqqg kmlrnlgsvk lepqqiqamr nlaqvkmepq hseqslflqg qqrqqqqqqq |
| 241 | qqflqmpgqs pqaqmnifqq qrlmqlqqqq llksmpqqrp qlpqqfqqqn lplrpplkpv |
| 301 | yepgmgaqrl tqymyrqqhr pednniefwr kfvaeyfapn akkrwcvsmy gsgrqttgvf |
| 361 | pqdvwhceic nrkpgrgfea taevlprlfk ikyesgtlee llyvdmpres qnssgqivle |
| 421 | yakatqesvf ehlrvvrdgq lrivfspdlk ifswefcarr heeliprrll ipqvsqlgsa |
| 481 | aqkyqqaaqn attdsalpel qnncnmfvas arqlakalev plvndlgytk ryvrclqise |
| 541 | vvnsmkdlid ysretrtgpi eslakfprrt gpssalpgps pqqasdqlrq qqqqqqqqqq |
| 601 | qqqqqqqqqq qqqtvsqntn sdqssrqval mqgnpsngvn yafnaasast stssiaglih |
| 661 | qnsmkgrhqn aaynppnspy ggnsvqmqsp sssgtmvpss sqqqhnlptf qsptsssnnn |
| 721 | npsqngipsv nhmgstnspa mqqagevdgn esssvqkiln eilmnnqahn nssggsmvgh |
| 781 | gsfgndgkgq anvnssgvll mngqvnnnnn tniggaggfg ggigqsmaan ginningnns |
| 841 | lmngrvgmmv rdpngqqdlg nqllgavngf nnfdwna |


SEQUENCE LISTING (20 sequences)

SEQ ID NO: 1 - DNA, 711 bp, Nicotiana tabacum
atgaatcaaccaatttatacagagttgccgccggcgaattttccgggagaatttccggtgtaccgccggaattcaagcttcagtcgtctaatcccatgtttaactgaaacatggggcgacttaccactaaaagtcgacgattctgaagatatggtaatttatactctcttaaaagacgctcttaacgtcggatggtcgccgtttaatttcagcgccggcgaagtaaaatcggagcagagggaggaagaaattgtggtttctccggcggagacgacggccgcgccggcggctgagttacctaggggaaggcattacagaggtgttagacgacggccgtgggggaaatttgcggcggagattagggatccggcgaagaatggagctagggtttggcttggaacatacgaaacagatgaagaagctgcaattgcttatgataaagcggcttatagaatgcgcggttcaaaggctcatttaaattttccacatagaatcggtttaaatgaaccggaaccggttcgagttacggcgaaaagacgagcatcgcctgaaccggctagttcgtcggaaaatagttcacctaaacggagaagaaaggctgttgcaactgagaaatctgaagcagtagaagtggagagtaaatcaaatgttttgcaaactggatgtcaagttgaactattgacacgtcgacatcaattattagtcagttaa

SEQ ID NO: 2 - DNA, 705 bp, Nicotiana tabacum
atgtcaagtaactcaagcccactagaaatagacacttcattttcacattccaacttcttctttctccaagatcaatcaccaattttacaatgggatgatgatcttttcttcaatgatccatggtttgatgatgatcaatcaccaattataccatgtaactcagagaaagatgaaaatcatcaagtatttgaagaatcctcagacaatacaatcatgtcaaaaggaagtagccatggtcaagaattagaagaggtaacatcccaagaagaaaaagaaaaagaagaagaagaaaaacactatataggagttagaaaaaggccatggggtaaatatgcagcagaaataagagattcaacaagaaatggaattagggtttggttagggacatttgatacagctgaagaagctgctttagcttatgatcaagctgcattatcgatgagaggtccttggtctcttcttaattttccattggagaaagtcaagaaatcacttgaaaaaattgagtattcttgtaaagatggattgtctcctgctgctgttctaaaagctactcataaaacaaggagagtgaagcataaaagaagtagtagaaagaaaaagaataaagaaactcataatgttattgtttttgaggacttgggtgctgagttattagaagagcttttaatgacttcatcacaacattcgtgtcgaagggactga

SEQ ID NO: 3 - DNA, 858 bp, Nicotiana tabacum
acggggggggggggggggggggacttgaagactgggaagctccattaacgagctccgacaactcaacagcctctgatttaagccgaagcaatagcattgagtccaacatgtttcctaattgcttgcccaatgaatataattatacagctgatatgttttttaacgatatctttaatgaaggcattgttggctatggatttgagccagcttctgaatttacactccccagtatcaaattggagccagaaatgactgtacaatcacctgcaatatggaatttaccggagtttgtggcgccgccggagacggcggcggaggtgaaactggaaccaccggcgccgcaaaaggcaaagcattataggggagtgagagtgaggccgtgggggaagtttgcagcggaaattagggatccggcaaagaatggggcaagggtgtggctgggtacgtatgagacggcagaggacgcagcgtttgcttatgacaaggcggcgtttcgcatgcgggggtcacgtgcattgcttaatttcccgttaaggattaattctggtgagcctgatcccattagagttggttctaaaaggtcatcaatgtcgccggagtattcttcttcttcatcgtcgtcggcgtcgtcgccgaagaggaggaagaaggtatctcaagggacggagctaacggtgttataggtcccaactgggttctgtgtagtgattaagaaaaatagaattagtcgagggaatttgttttttacttggctgaagtaatgaatttgttatttatttattttttgactgtggttgaaattgaatcaaaaaaaaaaaaaaaaaagtactagtcgacgcgtggcctagtagtagtaga

SEQ ID NO: 4 - DNA, 702 bp, Nicotiana tabacum
atgtatcaaccaatttcgaccgagctacctccgacgagtttcagtagtctcatgccatgtttgacggatacatggggtgacttgccgttaaaagttgatgattccgaagatatggtaatttatgggctcttaagtgacgctttaactgccggatggacgccgtttaatttaacgtccaccgaaataaaagccgagccgagggaggagattgagccagctacgattcctgttccttcagtggctccacctgcggagactacgacggctcaagccgttgttcccaaggggaggcattataggggcgttaggcaaaggccgtgggggaaatttgcggcggaaataagggacccagctaaaaacggcgcacgggtttggctagggacttatgagacggctgaagaagccgcgctcgcttatgataaagcagcttacaggatgcgcggctccaaggctctattgaattttccgcataggatcggcttaaatgagcctgaaccggttagactaaccgctaagagacgatcacctgaaccggctagctcgtcaatatcatcggctttggaaaatggctcgccgaaacggaggagaaaagctgtagcggctaagaaggctgaattagaagtgcaaagccgatcaaatgctatgcaagttgggtgccagatggaacaatttccagttggcgagcagctattagtcagttaa

SEQ ID NO: 5 - DNA, 924 bp, Nicotiana tabacum
atccagaattaataaaccctagtaagtgaaagtgaaagaaactactcatccaaatatctatagaaaagtaaatgaatcccgctaatgcaaccttctctttctctgagcttgatttccttcaatcaatagaaaaccatcttctgaattatgattccgatttttctgaaattttttcgccgatgagttcaagtaacgcattgcctaatagtcctagctcaagttttggcagcttcccttcagcagaaaatagcttggatacctctctttgggatgaaaactttgaggaaacaatacaaaatctcgaagaaaagtccgagtccgaggaggaaacaaaggggcatgtcgtggcgcgtgagaaaaacgcgacacaagattggagacggtacataggagttaaacggcggccgtgggggacgttttcggcggagataagggacccggagagaagaggcgcgagattatggctaggaacttacgagaccccagaggacgcagcattggcttacgatcaagccgctttcaaaatccgcggctcgagagctcggctcaattttcctcacttaattggatcaaacattcctaagccggctagagttacagcgagacgtagccgtacgcgctcaccccagccatcgtcttcttcatgtacctcatcatcagaaaatgggacaagaaaaaggaaaatagatttgataaattccatagccaaagcaaaatttattcgtcatagctggaacctacaaatgttgctataactgtatttaatttggaaggaattaattaaggttattctatgtctttgtattagaatttagaataattccctaaagctcctgaagaacgaaacttgtaaacatctctctgtctccgtatcatgttctaatttaacatgaaattacatgagcgcaaaaaaaaaaaaaaaaa

SEQ ID NO: 6 - DNA, 741 bp, Nicotiana tabacum
atgtatcaacccatttctacagaattcccagtatatcaccggacttcaagtttcagtagtctcatgccatgtttgacggatacttggggtgacttgccgttaaaagttgatgattccgaagatatggtaatttatgggctcttaagtgacgctttaactaccggatggacgccgtttaatttaacgtccaccgaaataaaagccgagccgagggaagagattgagccagctacgagtcctgttccttcagtggctccacccgcggagactacgacggctcaagccgtcgtgcccaagggaaggcattataggggcgtcaggcaaaggccgtgggggaaatttgcggcggaaataagggacccagctaaaaatggcgcacgggtttggctagggacttatgagacggctgaagaagccgcgctcgcttatgataaagcagcttacaggatgcgcggctccaaggctctattgaattttccgcataggatcggcttaaatgagcctgaaccggttaggctgaccgttaagagacgatcacctgaaccggccgttaagagacgatcacctgaaccggctagctcgtcaatatcaccggcttcggaaaatagcttgccgaagcggaggagaaaagctgtagcggctaagcaagctgaattagaagtgcagagccgatcaaatgtaatgcaagttgggtgccaaatggaacaatttccagttggcgagcagctattagttagttaa

SEQ ID NO: 7 - DNA, 2046 bp, Nicotiana tabacum
atgactgattacagcttacccaccatgaatttgtggaatactagtggtactaccgatgacaacgttactatgatggaagcttttatgtcttctgatctcacttcattttgggctacttctaattctactgctgttgctgctgttacctctaattctaatcatattccagttaataccccaacggttcttcttccgtcttcttgtgcctctactgtcacagctgtggctgtcgatgcttcaaaatccatgtcttttttcaaccaagaaacccttcaacagcgtcttcaaacgctcattgatggtgctcgtgagacgtggacctatgccatcttttggcagtcatccgccgttgatttaacgagtccgtttgtgttgggctggggagatggttactacaaaggtgaagaagataaagccaataggaaattagctgtttcttctcctgcttatatagctgagcaagaacaccggaaaaaggttctccgggagctgaattcgttgatttccggcacgcaaaccggcactgatgatgccgtcgatgaagaagttaccgacactgaatggttcttccttatttccatgacccagtcgtttgttaacggaagtgggcttccgggtcaggccttatacaattccagccctatttgggtcgccggagcagagaaattggcagcttcccactgcgaacgggctcggcaggcccagggattcgggcttcagacgatggtttgtattccttcagcaaacggcgtggttgaattgggctccacggagttgattattcagagttctgatctcatgaacaaggttagagtattgtttaacttcaataatgatttgggctctggttcgtgggctgtgcaacccgagagcgatccgtccgctctttggctcactgatccatcgtctgcagctgtacaagtcaaagatttaaatacagttgaggcaaattcagttccatcaagtaatagtagtaagcaagttgtatttgataatgagaataatggtcacagttgtgataatcagcaacagcaccattctcggcaacaaacacaaggattttttacaagggagttgaacttttcagaattcgggtttgatggaagtagtaataataggaatgggaattcatcactttcttgcaagccagagtcgggggaaatcttgaattttggtgatagcactaagaaaagtgcaaatgggaacttattttccggtcagtcccattttggtgcaggggaggagaataagaagaagaaaaggtcacctgcttccagaggaagcaatgaagaaggaatgctttcatttgtttcaggtacaatcttgcctgcagcttctggtgcgatgaagtcaagtggatgtgtcggtgaagactcctctgatcattcggatcttgaggcctcagtggtgaaagaagctgaaagtagtagagttgtagaacccgaaaagaggccaaagaagcgaggaaggaagccagcaaatggacgtgaggaacctttgaatcacgtcgaagcagagaggcaaaggagagagaaattaaaccaaaggttctacgctttaagagctgttgttccgaatgtgtccaagatggacaaggcatcactgcttggagatgcaatttcatatattaatgagctgaagttgaagcttcaaactacagaaacagatagagaagacttgaagagccaaatagaagatttgaagaaagaattagatagtaaagactcaaggcgccctggtcctccaccaccaaatcaagatcacaagatgtctagccatactggaagcaagattgtagatgtggatatagatgttaagataattggatgggatgcgatgattcgtatacaatgtaataaaaagaaccatccagctgcaaggttaatggtagccctcaaggagttagatctagatgtgcaccatgccagtgtttcagtggtgaatgatttgatgatccaacaagccacagtgaaaatgggtagcagactttacacggaagagcaacttaggatagcattgacatccagagttgctgaaacacgctaa

SEQ ID NO: 8 - DNA, 2148 bp, Nicotiana tabacum
cgcagacccctcttttcacccatttctctctctctctctctctctctctctatatatatatatatctttcacgccaccatatccaactgtttgtgctgggtttatggaatgactgattacagcttacccaccatgaatttgtggaatactagtggtactaccgatgacaacgtttctatgatggaatcttttatgtcttctgatctcacttcattttgggctacttctaattctactactgctgctgttacctctaattctaatcttattccagttaataccctaactgttcttcttccgtcttcttgtgcttctactgtcacagctgtggctgtcgatgcttcaaaatccatgtcttttttcaaccaagaaactcttcagcagcgtcttcaaaccctcattgatggtgctcgtgagacgtggacctatgccatcttttggcagtcatccgtcgttgatttatcgagtccgtttgtgttgggctggggagatggttactacaaaggtgaagaagataaagccaataggaaattagctgtttcttctcctgcttatattgctgagcaagaacaccgaaaaaaggttctccgggagctgaattcgttgatctccggcacgcaaaccggcactgatgatgccgtcgatgaagaagttaccgacactgaatggttcttccttatttccatgacccaatcgtttgttaacggaagtgggcttccgggtcaggccttatacaattccagccctatttgggtcgccggagcagagaaattggcagcttcccactgcgaacgggctcggcaggcccagggattcgggcttcagacgatggtttgtattccttcagcaaacggcgtggttgaattgggctccacggagttgataatccagagttgtgatctcatgaacaaggttagagtattgtttaacttcaataatgatttgggctctggttcgtgggctgtgcagcccgagagcgatccgtccgctctttggctcactgatccatcgtctgcagctgtagaagtccaagatttaaatacagttaaggcaaattcagttccatcaagtaatagtagtaagcaagttgtgtttgataatgagaataatggtcacagttctgataatcagcaacagcagcattctaagcatgaaacacaaggatttttcacaagggagttgaatttttcagaatttgggtttgatggaagtagtaataataggaatgggaattcatcactttcttgcaagccagagtcgggggaaatcttgaattttggtgatagtactaagaaaagtgcaaatgggaacttattttcgggtcagtcccattttggggcaggggaggagaataagaacaagaaaaggtcacctgcttccagaggaagcaatgaagaaggaatgctttcatttgtttcgggtacaatcttgcctgcagcttctggtgcgatgaagtcaagtggaggtgtaggtgaagactctgatcattcggatcttgaggcctcagtggtgaaagaagctgaaagtagtagagttgtagaacccgaaaagaggccaaagaagcgaggaaggaagccagcaaatggacgggaggaacctttgaatcacgtcgaagcagagaggcaaaggagagagaaattaaaccaaaggttctacgcattaagagctgttgttccgaatgtgtccaagatggacaaggcatcactgcttggagatgcaatttcatatattaatgagctgaagttgaagcttcaaaatacagaaacagatagagaagaattgaagagccaaatagaagatttaaagaaagaattagttagtaaagactcaaggcgccctggtcctccaccatcaaatcatgatcacaagatgtctagccatactggaagcaagattgtagacgtggatatagatgttaagataattggatgggatgcgatgattcgtatacaatgtaataaaaagaatcatccagctgcaaggttaatggtagccctcaaggagttagatctagatgtgcaccatgccagtgtttcagtggtgaacgatttgatgatccaacaagccactgtgaaaatgggtagcagactttacacggaagagcaacttaggatagcattgacatccagagttgctgaaacacgctaa

SEQ ID NO: 9 - DNA, 1980 bp, Nicotiana tabacum
atgacggattatagaataccaacgatgactaatatatggagcaatactacatccgatgataatatgatggaagcttttttatcttctgatccgtcgtcgttttggcccggaacaactactacaccaactccccggagttcagtttctccagcgccggcgccggtgacggggattgccggagacccattaaagtctatgccatatttcaaccaagagtcactgcaacagcgactccagactttaatcgatggggctcgcaaagggtggacgtatgccatattttggcaatcgtctgttgtggatttcgcgagcccctcggttttggggtggggagatgggtattataaaggtgaagaagataaaaataagcgtaaaacggcgtcgttttcgcctgactttatcacggaacaagcacaccggaaaaaggttctccgggagctgaattctttaatttccggcacacaaaccggtggtgaaaatgatgctgtagatgaagaagtaactgatactgaatggttttttctgatttccatgacacaatcgtttgttaacggaagcgggcttccgggcctggcgatgtatagttcaagcccgatttgggttactggaacagagagattagctgtttctcactgtgaacgggcccgacaggcccaaggtttcgggcttcagactattgtttgtattccttcagctaatggtgttgttgagctcgggtcaactgagttgatattccagactgctgatttaatgaacaaggttaaagttttgtttaattttaatattgatatgggtgcgactacgggctcaggatcgggctcatgtgctattcaggccgagcccgatccttcagccctttggctgactgatccggcttcttcagttgtggaagtcaaggattcgtcgaatacagttccttcaaggaataccagtaagcaacttgtgtttggaaatgagaattctgaaaatgttaatcaaaattctcagcaaacacaaggatttttcactagggagttgaatttttccgaatatggatttgatggaagtaatactcggtatggaaatgggaatgcgaattcttcgcgttcttgcaagcctgagtctggtgaaatcttgaattttggtgatagtactaagaggagtgcttgcagtgcaaatgggagcttgttttcgggccaatcacagttcgggcccgggcctgcggaggagaacaagaacaagaacaagaaaaggtcacctgcatcaagaggaagcaacgatgaaggaatcctttcatttgtttcgggtgtgattttgccaagttcaaacacggggaagtccggtggaggtggcgattcggatcaatcagatctcgaggcttcggtggtgaaggaggcggatagtagtagagttgtagaccccgagaagaagccgaggaaacgagggaggaaaccggctaacgggagagaggagccattgaatcatgtggaggcagagagacaaaggagggagaaattgaatcaaagattctatgcacttagagctgttgtaccaaatgtgtcaaaaatggataaagcatcacttcttggtgatgcaattgcatttatcaatgagttgaaatcaaaggttcagaattctgactcagataaagaggacttgaggaaccaaatcgaatctttaaggaatgaattagccaacaagggatcaaactataccggtcctcccccgtcaaatcaagaactcaagattgtagatatggacatcgacgttaaggtgatcggatgggatgctatgattcgtatacaatctaataaaaagaaccatccagccgcgaggttaatgaccgctctcatggaattggacttagatgtgcaccatgctagtgtttcagttgtcaacgagttgatgatccaacaagcgactgtgaaaatgggaagccggctttacacgcaagaacaacttcggatatcattgacatccagaattgctgaatcgcgatga

SEQ ID NO: 10 - DNA, 1977 bp, Nicotiana tabacum
atgacggactatagaataccaacgatgactaatatatggagcaatacaacatccgacgataacatgatggaagcttttttatcttctgatccgtcgtcgttttgggccggaacaaatacaccaactccacggagttcagtttctccggcgccggcgccggtgacggggattgccggagacccattaaagtcgatgccgtatttcaaccaagagtcgctgcaacagcgactccagacgttaatcgacggggctcgcgaagcgtggacttacgccatattctggcaatcgtctgttgtggatttcgtgagcccctcggtgttggggtggggagatggatattataaaggagaagaagacaagaataagcgtaaaacggcggcgttttcgcctgattttattacggagcaagaacaccggaaaaaagttctccgggagctgaattctttaatttccggcacacaaactggtggtgaaaatgatgctgtagatgaagaagtaacggatactgaatggttttttctgatttcaatgactcaatcgtttgttaacggaagcgggcttccgggcctggctatgtacagctcaagcccgatttgggttactggaagagaaagattagctgcttctcactgtgaacgggcccgacaggcccaaggtttcgggcttcagactatggtttgtattccttcagctaatggtgttgttgagctcgggtcaactgagttgatattccagagcgctgatttaatgaacaaggttaaaatcttgtttgattttaatattgatatgggcgcgactacgggctcaggttcgggctcatgtgctattcaggctgagcccgatccttcaaccctttggcttacggatccaccttcctcagttgtggaagtcaaggattcgtcgaatacagttccttcaagtaatagtagtaagcaacttgtgtttggaaatgagaattctgaaaatgttaatcaaaattctcagcaaacacaaggatttttcactagggagttgaatttttccgaatatggatttgatggaagtaatactaggagtggaaatgggaatgtgaattcttcgcgttcttgcaagcctgagtctggcgaaatcttgaattttggtgatagtactaagagaaatgcttcaagtgcaaatgggagcttgttttcgggccaatcgcagttcggtcccgggcctgcggaggagaacaagaacaagaacaagaaaaggtcacctgcatcaagaggaagcaatgaagaaggaatgctttcatttgtttcgggtgtgatcttgccaagttcaaacacggggaagtccggtggaggtggcgattcggatcattcagatctcgaggcttcggtggtgaaggaggcggatagtagtagagttgtagaccccgagaagaggccgaggaaacgaggaaggaaaccggctaacgggagagaggagccattgaatcatgtggaggcagagaggcaaaggagggagaaattgaatcaaagattctatgcacttagagctgttgtaccaaatgtgtcaaaaatggataaagcatcacttcttggtgatgcaattgcatttatcaatgagttgaaatcaaaggttcagaattctgactcagataaagatgagttgaggaaccaaattgaatctttaaggaatgaattagccaacaagggatcaaactataccggtcctccaccgccaaatcaagatctcaagattgtagatatggatatcgacgttaaagtcatcggatgggatgctatgattcgtatacaatctaataaaaagaaccatccagccgcgaggttaatggccgctctcatggaattggacttagatgtgcaccatgctagtgtttcagttgtcaacgagttgatgatccaacaagcgacagtgaaaatggggagccggctttacacgcaagagcagcttcggatatcattgacatccagaattgctgaatcgcgatga

SEQ ID NO: 11 - protein, 298 aa, Drosophila sp.
MMMNAFIEPAQHHLASYGLRMSPNTTASNSNAQQQQQQQLEMTQQQQQQQQQQQQQQQQDQESAAATAAAYQNSGYGHFNSYASRDFLLGRREAEYGVAGSAGQASAAADSMLFSGFPAQAAELGSGFGQHPFHSHHHHHQMRMGMADAYAAGHPYNHHGNFPTAAVHHPVVHHPSHHAMSAMHPAGAGAFLRYMRHQPASSASSVKQEMQCLWIDPDQPGLVPPGGRKTCNKVFHSMHEIVTHLTVEHVGGPECTTHACFWVGCSRNGRPFKAKYKLVNHIRVHTGEKPFACPHPGC

SEQ ID NO: 12 - protein, 12 aa, Arabidopsis sp.
LDLDLELRLGFA

SEQ ID NO: 13 - protein, 24 aa, Arabidopsis sp.
EGGMEKRSQLLDLDLNLPPPSEQA

SEQ ID NO: 14 - protein, 225 aa, Nicotiana tabacum
MAVKNKVSNGNLKGGNVKTDGVKEVHYRGVRKRPWGRYAAEIRDPGKKSRVWLGTFDTAEEAAKAYDTAAREFRGPKAKTNFPSPTENQSPSHSSTVESSSGENGVHAPPHAPLELDLTRRLGSVAADGGDNCRRSGEVGYPIFHQQPTVAVLPNGQPVLLFDSLWRAGVVNRPQPYHVTPMGFNGVNAGVGPTVSDSSSAVEENQYDGKRGIDLDLNLAPPMEF

SEQ ID NO: 15 - protein, 35 aa, Nicotiana tabacum
VGPTVSDSSSAVEENQYDGKRGIDLDLNLAPPMEF

SEQ ID NO: 16 - protein, 204 aa, Arabidopsis thaliana
MERSNSIELRNSFYGRARTSPWSYGDYDNCQQDHDYLLGFSWPPRSYTCSFCKREFRSAQALGGHMNVHRRDRARLRLQQSPSSSSTPSPPYPNPNYSYSTMANSPPPHHSPLTLFPTLSPPSSPRYRAGLIRSLSPKSKHTPENACKTKKSSLLVEAGEATRFTSKDACKILRNDEIISLELEIGLINESEQDLDLELRLGFA

SEQ ID NO: 17 - protein, 30 aa, Arabidopsis thaliana
NDEIISLELEIGLINESEQDLDLELRLGFA

SEQ ID NO: 18 - protein, 969 aa, Arabidopsis thaliana
MSQTNWEADKMLDVYIHDYLVKRDLKATAQAFQAEGKVSSDPVAIDAPGGFLFEWWSVFWDIFIARTNEKHSEVAASYIETQMIKAREQQLQQSQHPQVSQQQQQQQQQQIQMQQLLLQRAQQQQQQQQQQHHHHQQQQQQQQQQQQQQQQQQQQHQNQPPSQQQQQQSTPQHQQQPTPQQQPQRRDGSHLANGSANGLVGNNSEPVMRQNPGSGSSLASKAYEERVKMPTQRESLDEAAMKRFGDNVGQLLDPSHASILKSAAASGQPAGQVLHSTSGGMSPQVQTRNQQLPGSAVDIKSEINPVLTPRTAVPEGSLIGIPGRFAVLSVSFQLVKRVKCSISTSAHKNLLRINDWFPVSGSNQGSNNLTLKGWPLTGFDQLRSGLLQQQKPFMQSQSFHQLNMLTPQHQQQLMLAQQNLNSQSVSEENRRLKMLLNNRSMTLGKDGLGSSVGDVLPNVGSSLQPGGSLLPRGDTDMLLKLKMALLQQQQQNQQQGGGNPPQPQPQPQPLNQLALTNPQPQSSNHSIHQQEKLGGGGSITMDGSISNSFRGNEQVLKNQSGRKRKQPVSSSGPANSSGTANTAGPSPSSAPSTPSTHTPGDVISMPNLPHSGGSSKSMMMFGTEGTGTLTSPSNQLADMDRFVEDGSLDDNVESFLSQEDGDQRDAVTRCMDVSKGFTFTEVNSVRASTTKVTCCHFSSDGKMLASAGHDKKAVLWYTDTMKPKTTLEEHTAMITDIRFSPSQLRLATSSFDKTVRVWDADNKGYSLRTFMGHSSMVTSLDFHPIKDDLICSCDNDNEIRYWSINNGSCTRVYKGGSTQIRFQPRVGKYLAASSANLVNVLDVETQAIRHSLQGHANPINSVCWDPSGDFLASVSEDMVKVWTLGTGSEGECVHELSCNGNKFQSCVFHPAYPSLLVIGCYQSLELWNMSENKTMTLPAHEGLITSLAVSTATGLVASASHDKLVKLWK

SEQ ID NO: 19 - protein, 877 aa, Arabidopsis thaliana
MVPSEPPNPVGGGENVPPSILGGQGGAPLPSQPAFPSLVSPRTQFGNNMSMSMLGNAPNISSLLNNQSFVNGIPGSMISMDTSGAESDPMSNVGFSGLSSFNASSMVSPRSSGQVQGQQFSNVSANQLLAEQQRNKKMETQSFQHGQQQSMQQQFSTVRGGGLAGVGPVKMEPGQVSNDQQHGQVQQQQQKMLRNLGSVKLEPQQIQAMRNLAQVKMEPQHSEQSLFLQQQQRQQQQQQQQQFLQMPGQSPQAQMNIFQQQRLMQLQQQQLLKSMPQQRPQLPQQFQQQNLPLRPPLKPVYEPGMGAQRLTQYMYRQQHRPEDNNIEFWRKFVAEYFAPNAKKRWCVSMYGSGRQTTGVFPQDVWHCEICNRKPGRGFEATAEVLPRLFKIKYESGTLEELLYVDMPRESQNSSGQIVLEYAKATQESVFEHLRVVRDGQLRIVFSPDLKIFSWEFCARRHEELIPRRLLIPQVSQLGSAAQKYQQAAQNATTDSALPELQNNCNMFVASARQLAKALEVPLVNDLGYTKRYVRCLQISEVVNSMKDLIDYSRETRTGPIESLAKFPRRTGPSSALPGPSPQQASDQLRQQQQQQQQQQQQQQQQQQQQQQQQTVSQNTNSDQSSRQVALMQGNPSNGVNYAFNAASASTSTSSIAGLIHQNSMKGRHQNAAYNPPNSPYGGNSVQMQSPSSSGTMVPSSSQQQHNLPTFQSPTSSSNNNNPSQNGIPSVNHMGSTNSPAMQQAGEVDGNESSSVQKILNEILMNNQAHNNSSGGSMVGHGSFGNDGKGQANVNSSGVLLMNGQVNNNNNTNIGGAGGFGGGIGQSMAANGINNINGNNSLMNGRVGMMVRDPNGQQDLGNQLLGAVNGFNNFDWNA

SEQ ID NO: 20 - DNA, 39 bp, Artificial Sequence
  Description of Artificial Sequence Synthetic oligonucleotide
ctggatctggatctagaactccgtttgggtttcgcttaa